(19) United States
(12) Patent Application Publication
(10) Pub. No.: US 2010/0196516 A1
(43) Pub. Date: Aug. 5, 2010

(54) TREATMENT OF INFECTIONS BY CARBON MONOXIDE

(75) Inventors: Ligia S. Nobre, Fanhoes (PT); Joao D. Seixas, Odivelas (PT); Carlos C. Romao, Cascais (PT); Ligia M. Saraiva, Carnaxide (PT)

Correspondence Address: WOLF GREENFIELD & SACKS, P.C., 600 ATLANTIC AVENUE, BOSTON, MA, 02210-2206, US

(73) Assignee: ALFAMA-Investigacao e Desenvolvimento de produtos Farmaceuticos, Lda, Porto Salvo (PT)

(21) Appl. No.: 12/597,665

(22) PCT Filed: Apr. 24, 2008
(86) PCT No.: PCT/PT08/00017

§ 371 Date: Mar. 30, 2010

Related U.S. Application Data
(60) Provisional application No. 60/925,976, filed on Apr. 24, 2007.

(51) Int. Cl. A61K 33/00; A61K 31/555; C07F 11/00; A61P 31/04; A61P 31/12; A61P 31/10; A61P 33/06; A61P 33/02

(52) U.S. Cl. 424/699; 514/188; 546/12; 556/59; 514/492

(57) ABSTRACT

The invention relates to the use of carbon monoxide (CO) to treat infections. The invention also provides novel carbon monoxide releasing molecules (CORMs).

### FIELD OF THE INVENTION

The invention relates to the use of carbon monoxide to treat infections.

### BACKGROUND OF THE INVENTION

Despite significant advances in diagnosis and therapy, infections remain a major cause of morbidity and mortality throughout the world. Even with aggressive management, many patients with severe infections and sepsis develop complications and some die. Sepsis claims more than 200,000 lives in the United States annually (Angus, D.C. & Wax, R. S. (2001) Crit Care Med 29, S109-16). The negative health effects of infections provide a strong incentive to identify new treatments for infections.

Carbon monoxide (CO) is endogenously produced in the human body, mainly from the oxidation of heme catalyzed by heme oxygenase (HO) enzymes. The induction of HO and the consequent increase in CO production play important physiological roles in vasorelaxation and neurotransmission and in the immune system. The exogenous administration of CO gas and CO-releasing molecules (CORMs) has been shown to induce vascular effects and to alleviate hypoxia-reoxygenation injury of mammalian cells. In particular, due to its anti-inflammatory, antiapoptotic, and antiproliferative properties, CO inhibits ischemic-reperfusion injury and provides potent cytoprotective effects during organ and cell transplantation. Despite these findings regarding the physiology and biology of CO in mammals, nothing is known about the action of CO on microorganisms such as microbes that cause infections.

### SUMMARY OF THE INVENTION

This invention is based on the surprising discovery that CO caused cell death of three bacteria, Escherichia coli (E. coli), Staphylococcus aureus (S. aureus) and Helicobacter pylori (H. pylori), particularly when delivered through organometallic CORMs. These findings provide evidence that CO can be utilized as an anti-infective agent.

Without intending to be bound by any particular mechanism or theory, it is believed that CO may bind to transition metal-containing proteins in microorganisms (such as bacteria), giving rise to structural modifications and alterations of their biological functions and possibly accounting for the toxic effect of CO on the microorganisms.

Thus, the invention involves, in one aspect, the administration of CO to a subject to treat an infection. The use of a CO in the manufacture of a medicament for the treatment of infections is also contemplated. The CO may be in the form of a dissolved gas, which may or may not be trapped in a carrier complex. In some preferred embodiments, the CO is administered in the form of a prodrug, such as a CO releasing molecule (CORM). Numerous CORMS are described herein and are suitable for the practice of the present invention. The invention also involves, in one aspect, novel compositions of matter.

According to one aspect of the invention, a method for treating a subject having or at risk of having an infection is provided. The method comprises administering to a subject in need of such a treatment an effective amount of CO to treat the infection. In some important embodiments, the CO is in the form of a prodrug, such as a CORM. In important embodiments, the CORM is a an organometallic compound or an organic compound.

According to another aspect of the invention, a method for treating an infection is provided. The method comprises instructing a subject having or at risk of having an infection to take an effective amount of CO for the purpose of treating the infection. The subject may be instructed to take the effective amount of CO in the form of a CORM. In some embodiments, the subject is further instructed to take an anti-infective agent other than the CO.

According to another aspect of the invention, a method for treating a subject having or at risk of developing an infection is provided. The method comprises providing the subject with a package containing a CORM and providing the subject with indicia indicating that the CORM is for treating the infection. In some embodiments, the indicia is/are on a vial containing the CORM. In some embodiments, the indicia accompany the package containing the CORM.

According to yet another aspect of the invention, a medical treatment product is provided. The product comprises a package containing a CORM and indicia indicating that the CORM is for treating an infection. In some embodiments, the CORM is in a bottle. The indicia may be on a label on the bottle. In some embodiments, the package further contains an anti-infective agent other than a CORM.

According to another aspect of the invention, the use of a CORM in the manufacture of a medicament for the treatment of an infection is provided.

According to still another aspect of the invention, a compound having a structure:

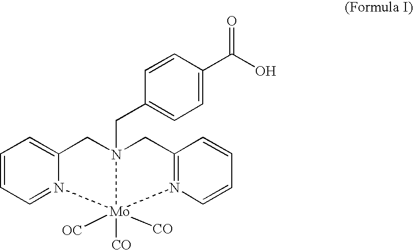

or a salt thereof is provided.

According to yet another aspect of the invention, a compound having a structure:

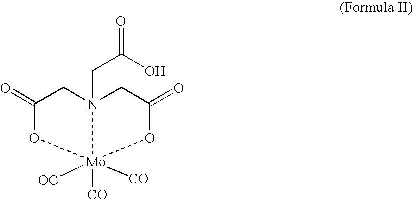

or a salt thereof is provided.

According to still another aspect of the invention, a compound having a structure:

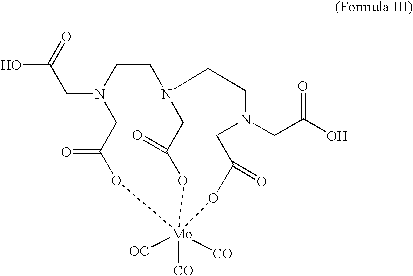

or a salt thereof is provided.

According to another aspect of the invention, a pharmaceutical composition is provided. The pharmaceutical composition comprises the compound of Formula I, the compound of Formula II, or the compound of Formula III and a pharmaceutically acceptable carrier. The pharmaceutical composition may further comprise one or more agents other than the compound of Formula I and/or the compound of Formula II and/or the compound of Formula III. In some embodiments, the agent may be an agent to treat an infection (e.g., an anti-infective agent). In some embodiments, the infection is caused by a bacterium. In some embodiments, the bacterium is Helicobacter pylori. Helicobacter pylori infection may cause gastritis, duodenal ulcer, gastric ulcer, stomach cancer, or non-ulcer dyspepsia.

In some embodiments, the agent is an antibiotic, an H₂-blocker, a proton pump inhibitor, a cytoprotective agent, or a combination thereof. The antibiotic may be metronidazole, tetracycline, amoxycillin, clarithromycin, furazolidone, ciproflaxin, rifabutin, or levoflaxin.

Examples of the H₂-blockers include cimetidine, famotidine, nizatidine, ranitidine, and ranitidine bismuth. The proton pump inhibitor may be omeprazole, lansoprazole, esomeprazole, pantoprazole, or rabeprazole. The cytoprotective agent may be bismuth subsalicylate, bismuth subcitrate, bismuth subnitrate, colloidal bismuth subcitrate, or sucralfate. In some embodiments, the agent is helidac, prevpac, or pylera.

According to still another aspect of the invention, a method of treating a subject having or at risk of developing a Helicobacter pylori infection is provided. The method comprises administering to a subject in need of such a treatment an effective amount of pharmaceutical composition comprising the compound of Formula I, the compound of Formula II, or the compound of Formula III and a pharmaceutically acceptable carrier to treat the infection. The Helicobacter pylori infection may cause gastritis, duodenal ulcer, gastric ulcer, stomach cancer, or non-ulcer dyspepsia.

The following embodiments apply equally to the various aspects of the invention set forth herein unless indicated otherwise.

In some preferred embodiments, the subject has an infection. In some embodiments, the subject is otherwise free of indications calling for treatment with CO.

In some embodiments, the CO is administered as a CORM. The CORM may be an organometallic compound or an organic compound. In some embodiments, the CORM is formulated in a pharmaceutically acceptable carrier that is an alginate solution.

The CORM may be administered orally, sublingually, buccally, intranasally, intravenously, intramuscularly, intrathecally, intraperitoneally, subcutaneously, intradermally, topically, rectally, vaginally, intrasynovially or intravitreously. In some preferred embodiments, the CORM is administered orally, intravenously, intramuscularly, or topically.

In some embodiments, the CORM is a compound having a structure:

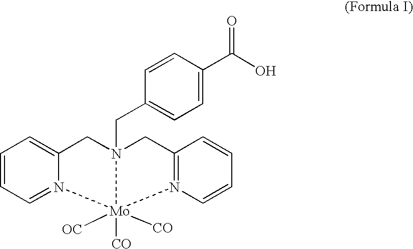

or a salt thereof, a compound having a structure:

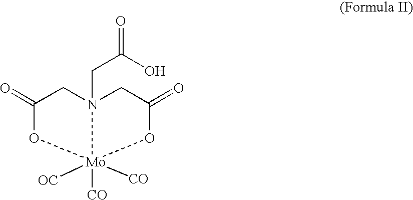

or a salt thereof, or a compound having a structure:

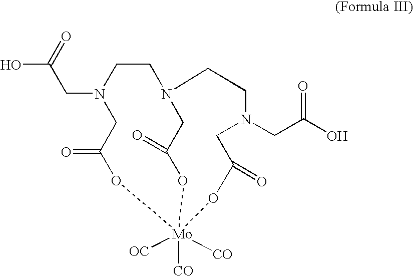

or a salt thereof, or a compound having a structure:

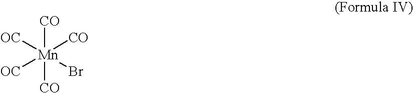

or a salt thereof, or a compound having a structure:

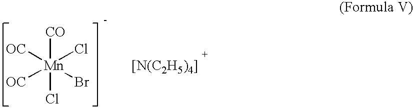

or a salt thereof, or a compound having a structure:

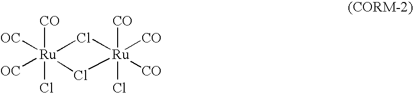

or a salt thereof, or a compound having a structure:

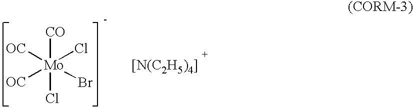

or a salt thereof.

The infection may be caused by a gram-positive bacterium, a gram-negative bacterium, an acid-fast bacillus, a spirochete, an actinomycete, a virus, a fungus, a parasite, Ureoplasma species, Mycoplasma species, Chlamydia species, or Pneumocystis species.

The gram-positive bacterium may be Staphylococcus species, Streptococcus species, Bacillus anthracis, Corynebacterium species, Diphtheroids species, Listeria species, Erysipelothrix species, or Clostridium species.

The gram-negative bacterium may be Helicobacter pylori, Neisseria species, Branhamella species, Escherichia species, Enterobacter species, Pasteurella species, Proteus species, Pseudomonas species, Klebsiella species, Salmonella species, Shigella species, Serratia species, Acinetobacter species, Haemophilus species, Brucella species, Yersinia species, Francisella species, Pasturella species, Vibrio cholera species, Flavobacterium species, Pseudomonas species, Campylobacter species, Bacteroides species, Fusobacterium species, Calymmatobacterium species, Streptobacillus species, or Legionella species.

The acid-fast bacillus may be a Mycobaterium species. Examples of spirochetes include Treponema species, Borrelia species, and Leptospira species.

The virus may be Retro virus, human immunodeficiency virus, Cytomegalovirus, Picorna virus, Polio virus, hepatitis A virus, enterovirus, Coxsackie virus, rhinovirus, echovirus, Calcivirus, Toga virus, equine encephalitis virus, rubella virus, Flavivirus, dengue virus, encephalitis virus, yellow fever virus, coronavirus, Rhabdovirus, vesicular stomatitis virus, rabies virus, Filovirus, ebola virus, Paramyxo virus, parainfluenza virus, mumps virus, measles virus, respiratory syncytial virus, Orthomyxovirus, influenza virus, Hantaan virus, bunga virus, phlebovirus, Nairo virus, Arena virus, hemorrhagic fever virus, reovirus, orbivirus, rotavirus, Birnavirus, Hepadnavirus, Hepatitis B virus, parvovirus, Papovavirus, papilloma virus, polyoma virus, Adenovirus, Herpes virus, varicella zoster virus, Pox viruses, variola virus, vaccinia virus, Iridovirus, African swine fever virus, delta hepatitis virus, non-A, non-B hepatitis virus, Hepatitis C, Norwalk virus, astrovirus, or unclassified virus.

Examples of fungi include Cryptococcus species, Histoplasma species, Coccidioides species, Paracoccidioides species, Blastomyces species, Chlamydia species, Candida species, Sporothrix species, Aspergillus species, and fungus of mucormycosis.

The parasite may be Plasmodium species, Toxoplasma species, Babesia species, Leishmania species, or Trypanosoma species.

In some preferred embodiments the infection is caused by Escherichia coli, Staphylococcus aureus, or Helicobacter pylori.

Each of the limitations of the invention can encompass various embodiments of the invention. It is, therefore, anticipated that each of the limitations of the invention involving any one element or combinations of elements can be included in each aspect of the invention. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

These and other aspects of the invention will be described in more detail below in connection with the detailed description of the invention.

All documents identified in this application are incorporated in their entirety herein by reference.

### BRIEF DESCRIPTION OF DRAWINGS

FIG. 1 shows the effects of CO gas on E. coli and S. aureus viability. (A) Histogram showing survival of E. coli and S. aureus. Cells were grown under microaerobic conditions in MS and LB media, respectively, and exposed to a flux of CO gas for 15 min. (See Materials and Methods in Example 1). (B) Sensitivity tests were conducted by plating the indicated serial dilutions of the cultures collected after 4 h of exposure to CO gas (+) or to nitrogen gas (−).

FIG. 2 shows the chemical structures of CORMs used in Example 1.

FIG. 3 shows the effects of CORM-2 on E. coli and S. aureus cell viability. (A) Histograms showing survival of E. coli and S. aureus. E. coli cells were grown in MS under aerobic and anaerobic conditions and treated with 250 μM CORM-2. S. aureus cells were grown aerobically and microaerobically in LB medium and exposed to 250 μM CORM-2. (B) Results of tests of the sensitivity of cultures to CORM-2 (see Materials and Methods in Example 1). The indicated dilutions of cultures were treated with CORM-2 (+; 250 μM) or left untreated (−) and assayed in the absence or in the presence of Hb.

FIG. 4 shows the effects of CORM-3 on E. coli and S. aureus cell viability. (A) Histograms showing survival of E. coli and S. aureus. E. coli cells were grown in MS medium either aerobically or anaerobically and treated with 400 μM CORM-3. S. aureus cells Were grown aerobically or microaerobically in LB medium to which 500 or 400 nM CORM-3 was added, respectively. (B) Sensitivity tests were conducted by plating dilutions of cultures grown as described in Materials and Methods of Example 1 after exposure to CORM-3 (+) or no treatment (−) in the absence or in the presence of Hb. The concentrations of CORM-3 used were the same as those indicated in the legend to panel A.

FIG. 5 shows the sensitivity of E. coli to compound of Formula IV and compound of Formula V. E. coli cells grown under aerobic or anaerobic conditions were treated with 500 or 200 μM compound of Formula IV, respectively, and with 50 μM compound of Formula V (see Materials and Methods in Example 1) in the absence or in the presence of Hb. The indicated dilutions of cultures exposed to CORMs (+) or not exposed (−) were subjected to sensitivity tests.

FIG. 6 shows the sensitivity of S. aureus to compound of Formula IV and compound of Formula V. S. aureus cells grown under aerobic and microaerobic conditions were treated with 600 μM compound of Formula IV and 50 μM compound of Formula V. The indicated dilutions of cultures exposed to CORMs (+) or not exposed (−) were subjected to sensitivity tests in the absence or in the presence of Hb, as described in Materials and Methods in Example 1.

FIG. 7 is a histogram showing the bactericial effect of CORM-2 on H. pylori survival. The paper disks were absorbed with 200 mM of CORM-2 and equal volume of DMSO was added to the control plates.

FIG. 8 is a histogram showing the bactericial effect of compound of Formula II and compound of Formula III on H. pylori survival. The paper disks were absorbed with 150 mM of each compound and the equal volume of water was added to the control plates.

### DETAILED DESCRIPTION OF THE INVENTION

The invention described herein relates, in part, to the use of CO for the treatment of infections. The invention also provides novel compositions of matter.

The present invention provides methods of treating an infection in a subject, comprising administering to the subject an effective amount of CO to treat the infection Preferably, the methods are employed to inhibit certain infections in a subject, such as a mammal. Methods of the invention also are readily adaptable for use in assay systems, e.g., assaying microbial replication and proliferation and properties thereof; as well as identifying compounds that affect microbes that cause infections.

As used herein the term “subject” means any mammal that may be in need of treatment. Subjects include but are not limited to: humans, non-human primates, cats, dogs, sheep, pigs, horses, cows, rodents such as mice, hamsters, and rats. Preferred subjects are human subjects.

The subject is known to have, is suspected of having been exposed, or is at risk of being exposed, or who has been exposed to an infection. In some preferred embodiments, the subject has an infection. The CO is administered in an amount effective to treat the infection in the subject.

In some embodiments, the subject is free of indications for treatment with CO.

CO (and CORMs) have been described for the treatment or prevention of diseases associated with inflammation and/or ischemia/reperfusion injury.

The term “treatment” or “treating” is intended to include prophylaxis, amelioration, prevention or cure of infections.

An “infection” or “infectious disease”, as used herein, refers to a disorder arising from the invasion of a host, superficially, locally, or systemically, by an infectious organism. Examples of infectious organisms include bacteria, viruses, parasites, fungi, and protozoa.

Bacteria include gram-negative and gram-positive bacteria. Examples of gram-positive bacteria include Pasteurella species, Staphylococcus species including Staphylococcus aureus, Streptococcus species including Streptococcus pyogenes group A, Streptococcus viridans group, Streptococcus agalactiae group B, Streptococcus bovis, Streptococcus anaerobic species, Streptococcus pneumoniae, and Streptococcus faecalis, Bacillus species including Bacillus anthracis, Corynebacterium species including Corynebacterium diphtheriae, aerobic Corynebacterium species, and anaerobic Corynebacterium species, Diphtheroids species, Listeria species including Listeria monocytogenes, Erysipelothrix species including Erysipelothrix rhusiopathiae, Clostridium species including Clostridium perfringens, Clostridium tetani, and Clostridium difficile.

Gram-negative bacteria include Neisseria species including Neisseria gonorrhoeae and Neisseria meningitidis, Branhamella species including Branhamella catarrhalis, Escherichia species including Escherichia coli, Enterobacter species, Proteus species including Proteus mirabilis, Pseudomonas species including Pseudomonas aeruginosa, Pseudomonas mallei, and Pseudomonas pseudomallei, Klebsiella species including Klebsiella pneumoniae, Salmonella species, Shigella species, Serratia species, Acinetobacter species; Haemophilus species including Haemophilus influenzae and Haemophilus ducreyi, Brucella species, Yersinia species including Yersinia pestis and Yersinia enterocolitica, Francisella species including Francisella tularensis, Pasturella species including Pasteurella multocida, Vibrio cholerae, Flavobacterium species, meningosepticum, Campylobacter species including Campylobacter jejuni, Bacteroides species (oral, pharyngeal) including Bacteroides fragilis, Fusobacterium species including Fusobacterium nucleatum, Calymmatobacterium granulomatis, Streptobacillus species including Streptobacillus moniliformis, Legionella species including Legionella pneumophila.

Other types of bacteria include acid-fast bacilli, spirochetes, and actinomycetes.

Examples of acid-fast bacilli include Mycobacterium species including Mycobacterium tuberculosis and Mycobacterium leprae.

Examples of spirochetes include Treponema species including Treponema pallidum, Treponema pertenue, Borrelia species including Borrelia burgdorferi (Lyme disease), and Borrelia recurrentis, and Leptospira species.

Examples of actinomycetes include: Actinomyces species including Actinomyces israelii, and Nocardia species including Nocardia asteroides.

Examples of viruses include but are not limited to: Retroviruses, human immunodeficiency viruses including HIV-1, HDTV-III, LAVE, HTLV-III/LAV, HIV-III, HIV-LP, Cytomegaloviruses (CMV), Picornaviruses, polio viruses, hepatitis A virus, enteroviruses, human Coxsackie viruses, rhinoviruses, echoviruses, Calciviruses, Togaviruses, equine encephalitis viruses, rubella viruses, Flaviruses, dengue viruses, encephalitis viruses, yellow fever viruses, Coronaviruses, Rhabdoviruses, vesicular stomatitis viruses, rabies viruses, Filoviruses, ebola virus, Paramyxoviruses, parainfluenza viruses, mumps virus, measles virus, respiratory syncytial virus (RSV), Orthomyxoviruses, influenza viruses, Bungaviruses, Hantaan viruses, phleboviruses and Nairo viruses, Arena viruses, hemorrhagic fever viruses, reoviruses, orbiviruses, rotaviruses, Birnaviruses, Hepadnaviruses, Hepatitis B virus, parvoviruses, Papovaviridae, papilloma viruses, polyoma viruses, Adenoviruses, Herpesviruses including herpes simplex virus 1 and 2, varicella zoster virus, Poxviruses, variola viruses, vaccinia viruses, Irido viruses, African swine fever virus, delta hepatitis virus, non-A, non-B hepatitis virus, Hepatitis C, Norwalk viruses, astroviruses, and unclassified viruses.

Examples of fungi include, but are not limited to: Cryptococcus species including Crytococcus neoformans, Histoplasma species including Histoplasma capsulatum, Coccidioides species including Coccidiodes immitis, Paracoccidioides species including Paracoccidioides brasiliensis, Blastomyces species including Blastomyces dermatitidis, Chlamydia species including Chlamydia trachomatis, Candida species including Candida albicans, Sporothrix species including Sporothrix schenckii, Aspergillus species, and fungi of mucormycosis.

Other infectious organisms include parasites. Parasites include Plasmodium species, such as Plasmodium species including Plasmodium falciparum, Plasmodium malariae, Plasmodium ovale, and Plasmodium vivax and Toxoplasma gondii. Blood-borne and/or tissues parasites include Plasmodium species, Babesia species including babesia microti and Babesia divergens, Leishmania species including Leishmania tropica, Leishmania species, Leishmania braziliensis, Leishmania donovani, Trypanosoma species including Trypanosoma gambiense, Trypanosoma rhodesiense (African sleeping sickness), and Trypanosoma cruzi (Chagas' disease).

Other medically relevant microorganisms have been described extensively in the literature, e.g., see C. G. A Thomas, Medical Microbiology, Bailliere Tindall, Great Britain 1983, the entire contents of which is hereby incorporated by reference.

There are various methods known in the art for administering CO. The CO may be administered, for example, as a gas, as a gas dissolved in a liquid or trapped in a carrier, or as a carbon monoxide releasing molecule (CORM). In some preferred embodiments, the CO is administered as a CORM.

CO delivered as a gas is described, for example, in WO 2003/000114 A3, US 2002/0155166 A1, WO 2004/043341 A2, US 2004/0052866 A1, WO 2003/072024 A2, US 2003/0219496 A1, WO 2003/103585 A2 and US 2005/0048133 A1.

As used herein, a CORM means a molecule having the ability to release carbon monoxide in vivo. Examples of such molecules are molecules containing CO and include a molecule that comprises CO. Other examples of CORMs are molecules capable of generating CO. CO can be released in certain conditions (e.g. oxidative conditions of a targeted site). Therapeutic delivery of CO by CORMs is described in WO 2005/013691 A1, US 2003/068387A1, WO 2004/0445599, WO 2003/066067A2, US 2004/067261A1, and U.S. Pat. No. 7,011,854. Therapeutic delivery of CO by heme containing carrier proteins is described in WO9422482.

In some embodiments, the CORM is a compound having a structure:

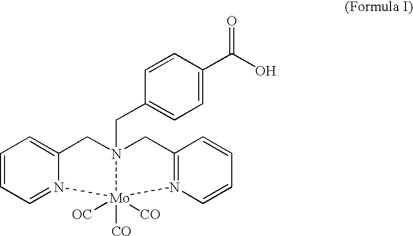

or a salt thereof, a compound having a structure:

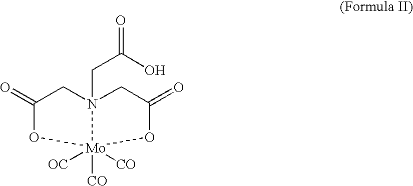

or a salt thereof, or a compound having a structure:

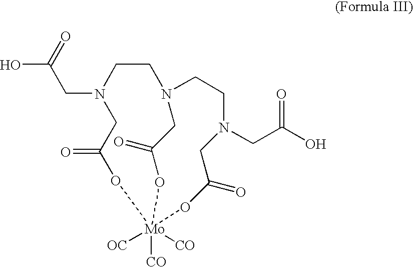

or a salt thereof, or a compound having a structure:

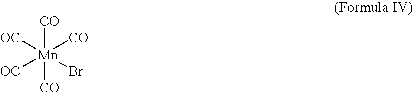

or a salt thereof, or a compound having a structure:

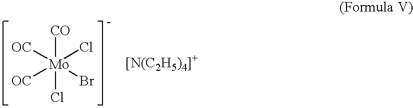

or a salt thereof, or a compound having a structure:

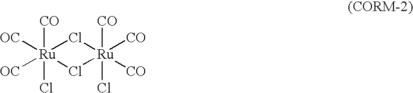

or a salt thereof, or a compound having a structure:

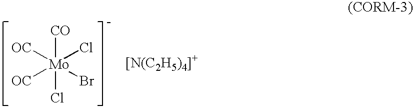

or a salt thereof.

Examples of CORMs include compounds from one of the following classes:

Class 1—CO containing organometallic complex. Such a compound can be dissolved in physiologically compatible support.

Class 2—CO containing organometallic complex linked to at least another pharmacologically important molecule. For example, said pharmacologically important molecule is a carrier or a drug. Furthermore, the CO containing organometallic complex and the at least other pharmacologically important molecule are optionally linked by means of an appropriate spacer.

Class 3—Supramolecule aggregates made of CO containing organometallic complexes optionally encapsulated e.g. in a cyclodextrin host and/or another appropriate inorganic or organic support.

Class 4—CO containing inorganic complex bearing ligands, e.g., polidentate ligands, containing N and/or S donors that function as reversible CO carriers.

Class 5—CO containing inorganic complex bearing ligands, e.g. polidentate ligands, containing N and/or S donors that function as reversible CO carriers, linked to at least another pharmacologically important molecule. For example, the pharmacologically important molecule is a carrier or a drug. Furthermore, the CO containing organometallic complex and the at least other pharmacologically important molecule are optionally linked by means of an appropriate spacer.

Class 6—Organic substances that release CO either by an enzymatic process or by decarbonylation. Such a compound can be dissolved in physiologically compatible supports.

Class 7—Organic substances that release CO either by an enzymatic process or by decarbonylation, e.g., dichloromethane optionally encapsulated either in cyclodextrin hosts and/or other appropriate inorganic or organic supports.

### Class 1—CO Containing Organometallic Complexes Dissolved in Physiologically Compatible Supports

This class of compounds comprises either simple 18 electron organometallic carbonyl complexes or modifications thereof designed to improve either their solubility in physiological media or their compatibility with membranes and biomolecules or tissues. The metals that may be used include first transition row biologically active metals (V, Cr, Mn, Fe, Co, Ni, Cu) as well as second (Mo, Ru, Rh, Pd) and third row elements (W, Re, Pt, Au), that appropriately bind the CO ligand. A large number of these compounds bears the cyclopentadienyl ligand (Cp) or derivatives thereof (indenyl, CpR5, and the like) hereby abbreviated as CpR(X), which enable the above-mentioned modifications, and impart some steric protection to the metal center with the corresponding higher reactivity control. The oxidation state of the metal in most of the complexes resembles the one usually found under biological conditions thereby facilitating later metabolization, after CO release.

In the examples listed immediately below, the term “pseudo-halide” is a general name given to mono-anionic ligands isoelectronic with the halides, e.g., thiocyanates, cyanates, cyanides, azides, etc. The term “hydrocarbyl chain” is the general name of a hydrocarbon radical comprising aliphatic CH₂ and/or aromatic residues, e.g., (CH₂)ₙ, n=2, 3, etc. or (CH₂)ₙ, (C₆H₄)ₘ, C₆H₅CH₂, etc. Alkyl is the general name given to the radical of an aliphatic hydrocarbon chain, e.g. methyl, ethyl, etc. Aryl is the general name given to a radical of an aromatic ring, e.g., phenyl, tolyl, xylyl, etc.

### Examples:

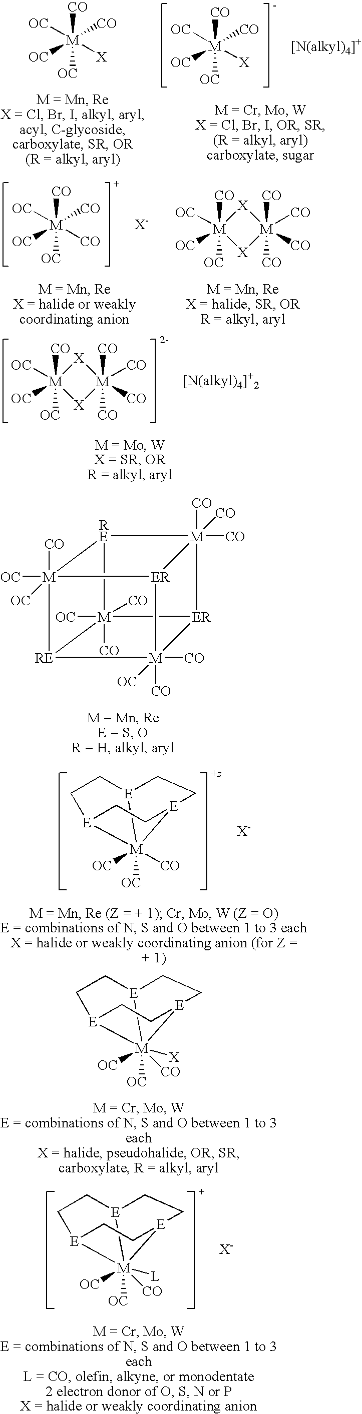

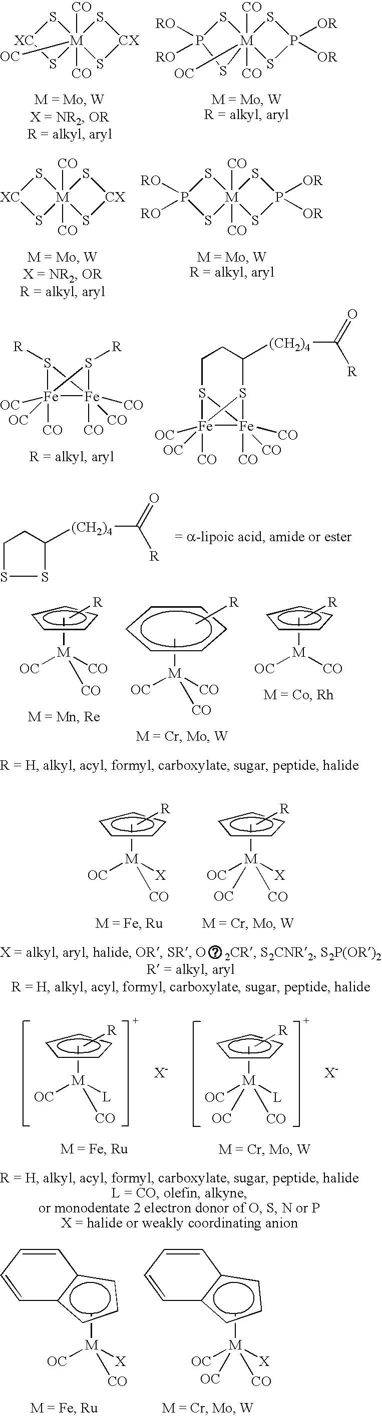

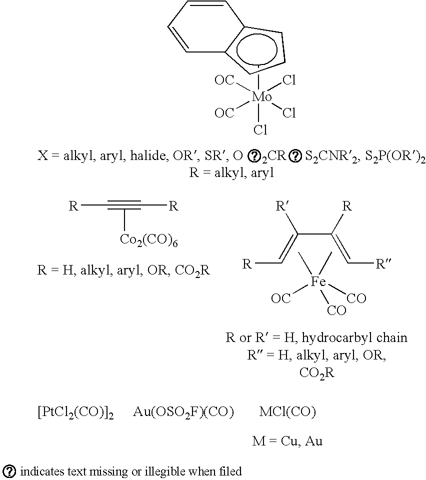

Several modifications can be envisaged to improve higher biological compatibility and solubility. One preferred possibility is to attach carboxylic, peptide or sugar derivatives to the cyclopentadienyl moiety. Examples are depicted for one Mn complex; similar derivatives can be made with compounds containing other metals, as well as for indenyl and other CpR(X) derivatives.

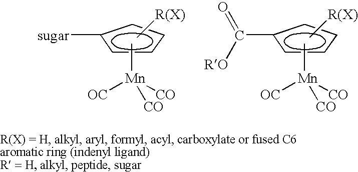

Further embodiments of Class 1 compounds, include:

[Mo(CO)₅Y]Q

wherein Y is bromide, chloride or iodide; and

### Q is [NR¹⁻⁴]⁺

where R¹, R², R³, and R⁴ are each independently alkyl.

As used herein, the term “alkyl” means a C₁-C₁₂ saturated hydrocarbon chain, such as methyl, ethyl, n-propyl, isopropyl, n-butyl, isobutyl, t-butyl, n-pentyl, n-hexyl, n-heptyl, n-octyl, n-nonyl, n-decyl, n-undecyl, or n-dodecyl. In one embodiment, alkyl is a C₁-C₆ or a C₁-C₄ saturated hydrocarbon chain.

Other embodiments of Class 1 compounds include:

[Mo(CO)₅Y]Q

wherein Y is bromide, chloride or iodide; and
Q is [NR₄]⁺, free or complexed with one cyclic polyether molecule or one or more acyclic polyether molecules, or
Na⁺, K⁺, Mg²⁺, Ca²⁺ or Zn²⁺, where each is free or complexed with one cyclic polyether molecule or one or more acyclic polyether molecules,
wherein
   each R is independently H or alkyl.

The cyclic polyether molecule includes, without limitation, crown ethers. In some embodiments, the cyclic polyether includes crown ethers from the 18-crown-6 family or the 15-crown-5 family. The one or more acyclic polyethers are of the polyethylene glycol type and of the formula R¹O(CH₂CH₂O)ₙR² where R¹ and R² are each independently H or alkyl and n is greater than or equal to 1. The acyclic polyether molecules are within the range of pharmaceutically acceptable polyethylene glycols or mono- or dialkyl polyethylene glycols.

When Q is free, Q is not associated with any molecular structure other than a molybdenum complex or molybdenum complexes by electrostatic (ionic) forces. When Q is complexed with one cyclic polyether molecule, or one or more acyclic polyether molecules, these complexed cationic entities are associated with one or more molybdenum anionic complexes by electrostatic bonding. When Q is complexed with acyclic polyethers, an ionic structure results from the interaction between the molybdenum complex or molybdenum complexes and the complexes formed between the acyclic polyethers and the NR₄⁺ or metal cation. The NR₄⁺ or metal cation may accommodate a variable, yet definite and controllable, number of non-covalently bound acyclic polyether molecules giving rise to different polymorphs or solvates. In one embodiment, the NR₄⁺ or metal cation non-covalently binds up to twelve acyclic polyether molecules at one time.

As used herein, the term “alkyl” means a C₁-C₁₂ saturated hydrocarbon chain, such as methyl, ethyl, n-propyl, isopropyl, n-butyl, isobutyl, t-butyl, n-pentyl, n-hexyl, n-heptyl, n-octyl, n-nonyl, n-decyl, n-undecyl, or n-dodecyl. In one embodiment, alkyl is a C₁-C₈ or a C₁-C₆ or a C₁-C₄ saturated hydrocarbon chain.

In some embodiments, Q is complexed with one cyclic polyether molecule or one or more acyclic polyether molecules. In some embodiments, the one cyclic polyether molecule includes crown ethers from the 18-crown-6 family or the 15-crown-5 family. In other embodiments Q is complexed by one or more acyclic polyethers in a coordination sphere comprising from four to twelve oxygen atoms of the ethyleneglycol or polyethylene glycol type chains. In yet another embodiment, Q is complexed by six, eight, or twelve acyclic polyether molecules. In another embodiment, Q is complexed by three acyclic diethers. In further embodiments, Q is complexed with one, two, or three polyether molecules.

In some embodiments, Q is complexed with more than one acyclic polyether molecules of the formula R¹O(CH₂CH₂O)ₙR² where R¹ and R² are each independently H or alkyl, n is greater than or equal to 1, and the polyether molecules are within the range of pharmaceutically acceptable polyethylene glycols or mono- or dialkyl polyethylene glycols. In further embodiments, when Q is complexed with more than one ether of the formula R¹O(CH₂CH₂O)ₙR², each R¹ and R² of each polyether molecule is independently H or alkyl, so that each polyether of the formula R¹O(CH₂CH₂O)ₙR² may be different and each R¹ or R² may be different than an R¹ or R² in another polyether molecule.

In further embodiments, specific acyclic ethers include, without limitation, monoglyme, diglyme, triglyme, PEG 400, PEG 1000, PEG 2000, PEG 3000 and PEG 4000, and methylPEG400.

Examples of the foregoing compounds include:

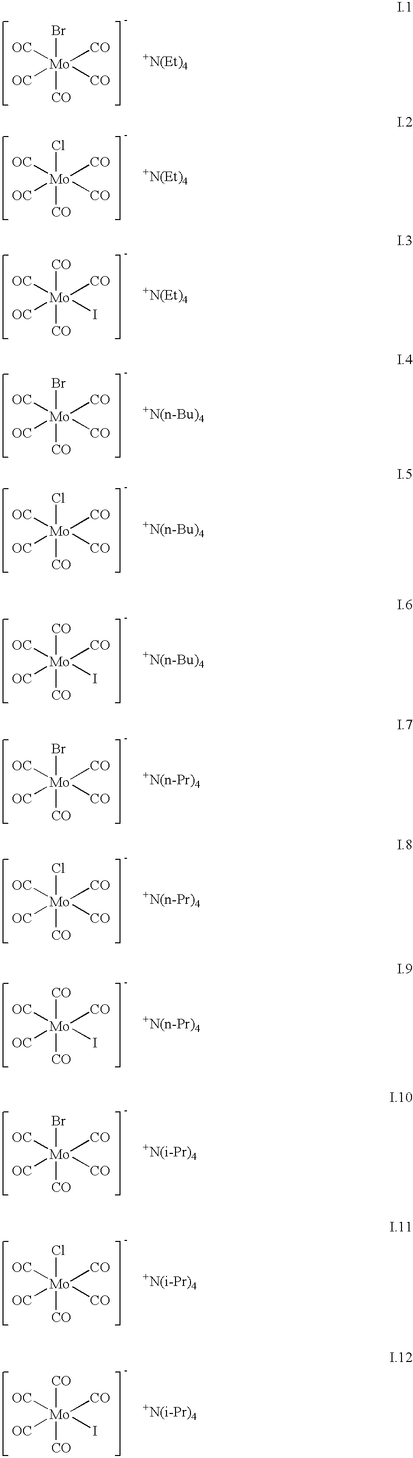

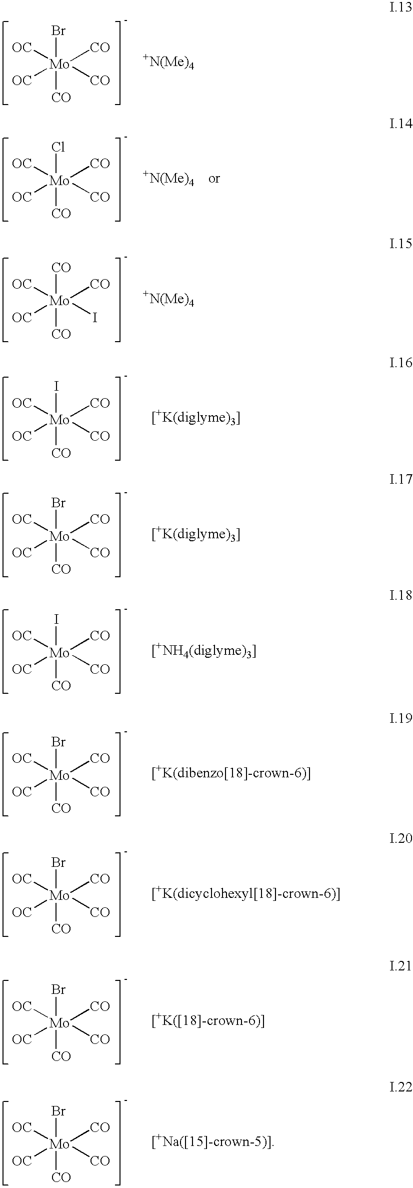

### Class 2—CO Containing Organometallic Complexes Linked to Other Pharmacologically Important Molecules.

This class of compounds takes advantage of the synergistic effects arising from the combination of two biologically active molecules, which both have beneficial effects. Examples for such drug-drug conjugates have been described in U.S. Pat. No. 6,051,576.

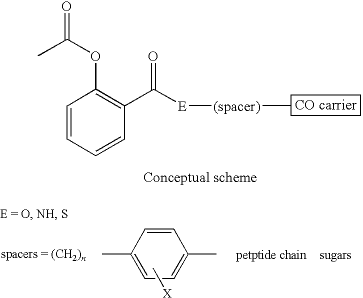

The above mentioned spacers comprise a variety of functions under the following specifications: the value of “n” in the linear hydrocarbon chain is an integer more specifically 1, 2, 3, 4: X is a general symbol for a substituent at the aromatic ring, namely, alkyl, aryl, alkoxy, aryloxl, halogen atom, thiolate; “peptide chain” represents a short chain of natural amino acids ranging from 1 to 4; by “sugars” it is meant the use of a mono-, di- or polysaccharide either protected or modified with adequate protection to increase lipophilicity and/or assure chemical stability of the drug-drug conjugate molecule, for example, with protective groups, such as esters, acetals, and silyl derivatives.

The definition of X given immediately above can be extended to carboxylates and amino acids in the cases where X is directly bound to the metal as in some of the examples depicted in the next scheme.

### Examples:

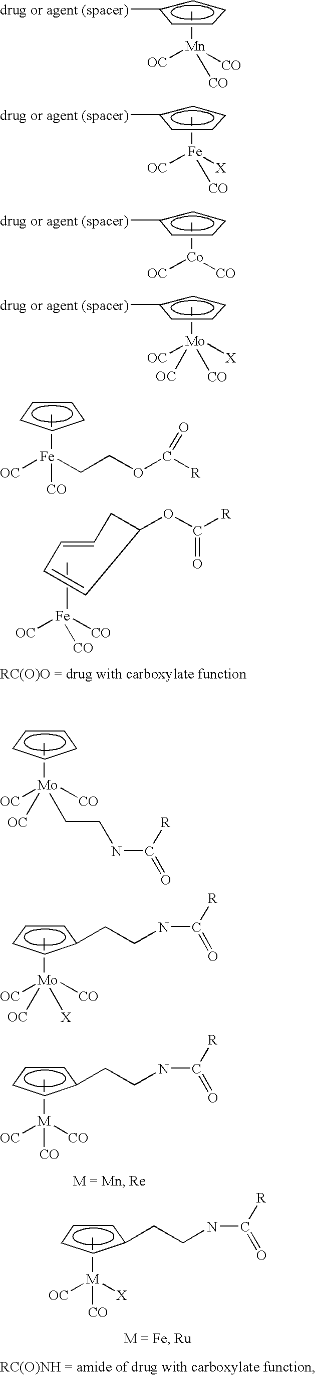

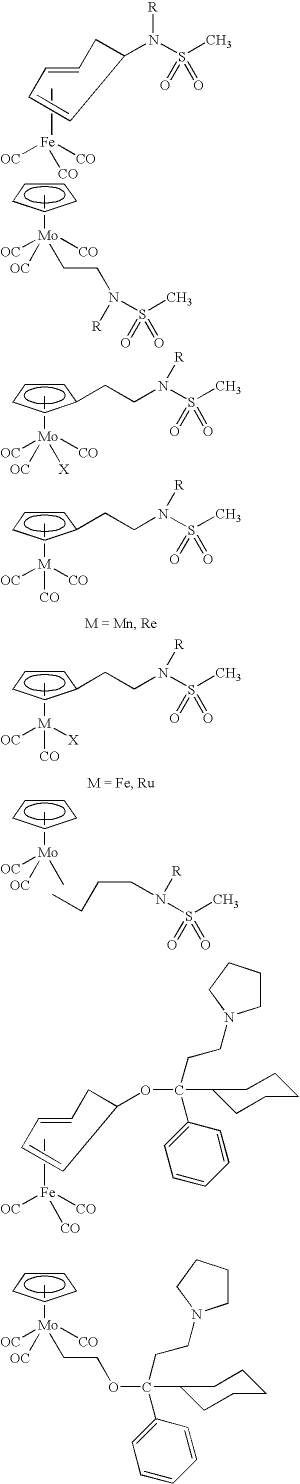

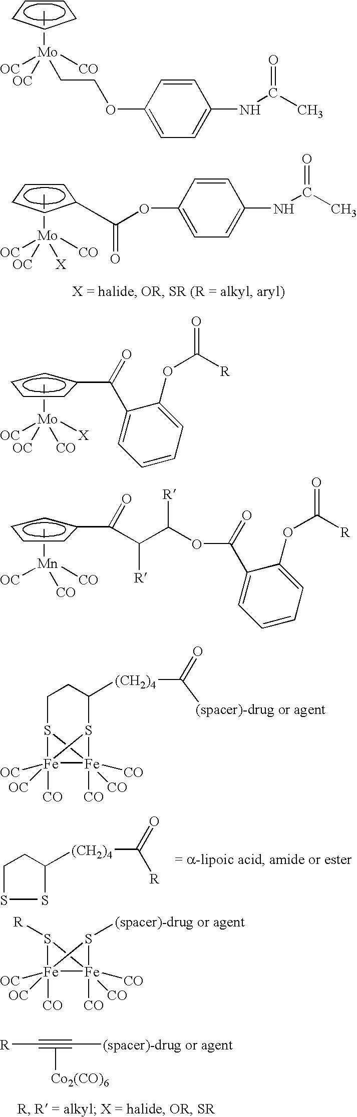

A second group of compounds bears the bioactive molecule bound directly to the metal, which can be achieved in several different manners as schematized below for the case of some iron and molybdenum cyclopentadienyl carbonyls, among others. The term “hydrocarbyl chain” is the general name of a hydrocarbon radical comprising aliphatic CH₂ and/or aromatic residues, e.g., (CH₂)ₙ, n=2, 3, etc. or (CH₂)ₙ, (C₆H₄)ₘ,

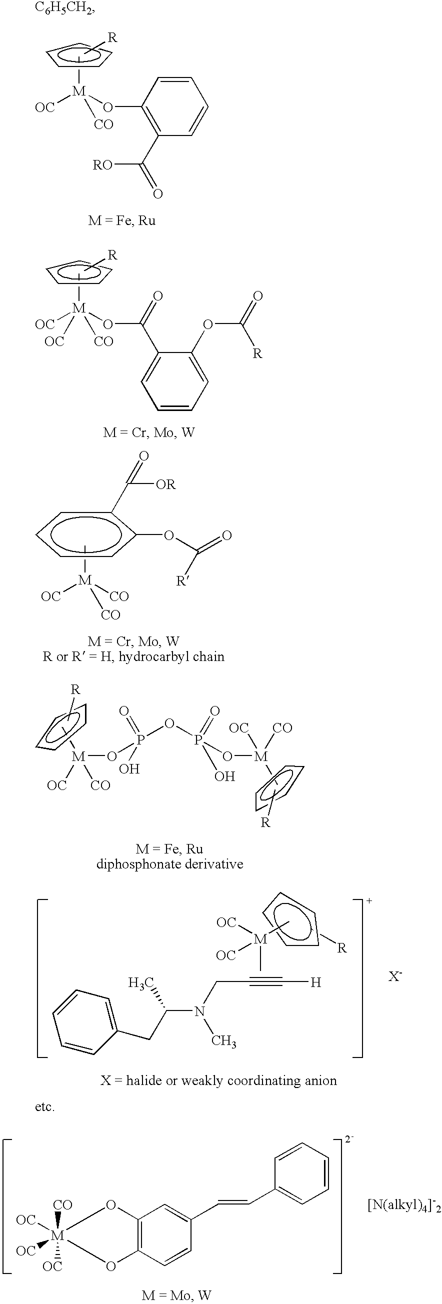

### Class 3: Encapsulated Supramolecular Aggregates Made of CO Containing Organometallic Complexes.

Controlled delivery of drugs into the organism is an important issue, especially in the case of drugs, which have undesired toxic effects if present systemically or at high local concentrations. CO release is a potential problem inasmuch as it can be toxic at high concentrations (see above). For certain applications, a slow release of CO in the blood or in specific target tissues is desirable. Encapsulation within host molecules that are non-toxic is one way to achieve a sustained release of active drugs in the organism. This strategy minimizes the undesired effects that may result from abrupt increases in the concentration and/or availability of a potentially toxic drug.

Cyclodextrins are well known hosts for many drugs and organic molecules and, recently have been applied to host organometallic molecules and enhance their delivery through physiological barriers or membranes. In this respect cyclodextrin has been found to be beneficial for increasing delivery of lipophilic drugs at the skin barrier. [T. Loftsson, M. Masson, Int. J. Pharm. 2001, 225, 15]. Cyclodextrin mediated supramolecular arrangements protect organometallic molecules for prolonged time periods and mask their reactivity, thereby, increasing their selectivity towards specific reagents. The hydrophobic part of carbonyl complexes as those exemplified under Class 1 above, fit inside β- or γ-cyclodextrin, or similar structures, with the CO groups facing the reaction medium and the organic ligands buried in the cavity. The resulting reduction in reactivity allows for the extension of the range of therapeutic CO-releasing complexes to cationic and anionic ones. Such charged complexes are more reactive and lose CO faster than the neutral ones when unprotected.

Liposomes and other polymeric nanoparticle aggregates are also useful carriers to target the delivery of CO-releasing organometallic complexes and the combined use of cyclodextrins with such aggregates has been considered as a very promising possibility for drug release. [D. Duchene, G. Ponchel, D. Wouessidjewe, Adv. Drug Delivery Rev. 1999, 36, 29.]

### CONCEPTUAL EXAMPLES

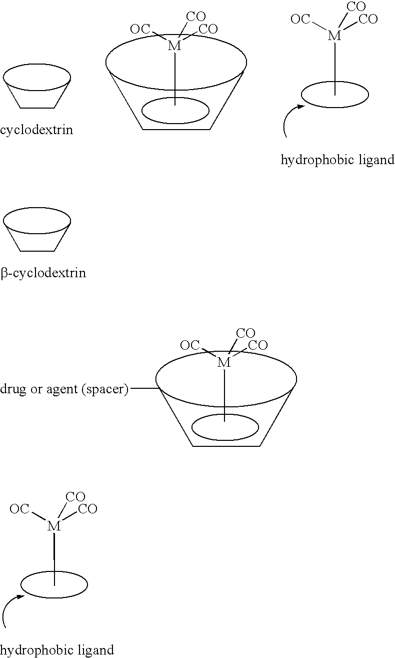

The actual examples cover organometallic molecules as (C₆H₆₋ₓRₓ)M(CO)₃ (M=Cr, Mo, W); (CpR₅)M(CO)₃X (M=Cr, Mo, W); (CpR₅)M(CO)₂X (M=Fe, Ru); (CpR₅)M(CO)₂ (M=Co, Rh) where R represents H, alkyl or other small functional group like methoxide, halide, carboxylic esters.

Mesoporous materials are chemically inert three dimensional molecules with infinite arrays of atoms creating channels and cavities of well defined pore size. These molecules are well suited to host organic and organometallic molecules in their pores. In the presence of biological fluids, smaller molecules undergoing acid-base and/or polar interactions with the inner walls of the pores slowly displace the included drugs, resulting in a controlled delivery of the active principle. Such aggregates have been prepared from M418 materials using organometallic molecules like those depicted under system 1 above. Examples include MCM-41 (linear tubes) and MCM-48 (cavities and pores)

Class 4—CO Containing Inorganic Complexes Bearing Ligands Containing N and/or S Donors that Function as Reversible CO Carriers.

Classical inorganic complexes bearing macrocyclic ligands on an equatorial plane of an octahedral coordination sphere are known to reversibly bind CO much in the same way as hemoglobin. The capacity to bind CO can be “tuned” by the nature of both the macrocycle and the ancilliary ligand trans to CO. A similar behavior has also been reported for other Fe(II) complexes bearing ligands that are much simpler than the porphyrin macrocycles that are the CO acceptor sites in hemoglobin and other heme containing proteins. In order to develop suitable CO delivering drugs, the later type of non-hemic complexes was chosen to avoid interference with the biological heme carriers, heme metabolism, and potential toxicity of heme or heme-like molecule. The complexes selected bear bidentate N donors (diamines, diglyoximes) or bidentate N,S donors of biological significance, like aminothiols or cysteine. Ancilliary ligands are N donors also of biological significance like imidazole, hystidine, and others. The complexes are soluble in aqueous media.

In the examples immediately below, the term pyridines refers to derivatives of the C₅H₅N ring (pyridine) bearing alkyl (R), alkoxy (OR), carboxy (C(O)OR), nitro (NO₂), halogen (X), substituents directly bound to the one or more positions of the C5 carbon ring, e.g. CH₃C₅H₄N, O₂NC₅H₄N. Amino-thiols refers to compounds bearing both the NH₂ (amino) and SH (thiol) functions bound to a hydrocarbon skeleton, e.g. H₂NCH₂CH₂SH, 1,2-C₆H₄(NH₂)(OH). A similar definition applies to amino alcohols, whereby the SH function is replaced by the OH (alcohol) function. The term amino acids refers to naturally occurring single amino acids coordinated in a bidentate fashion by the NH₂ and the COO functions as schematically depicted. Glyoximes are bidentate N donors, bearing either alkyl or aryl substituents on the hydrocarbon chain binding the two N atoms, as depicted in the first example below for a diaryl glyoxime. Diimines present a similar structure whereby the OH groups in the diglyoximes are replaced by alkyl or aryl groups. An extension of this family of ligands includes also 2,2′-bypiridines, e.g., 2,2′-dipyridyl, and phenanthrolines.

### Examples:

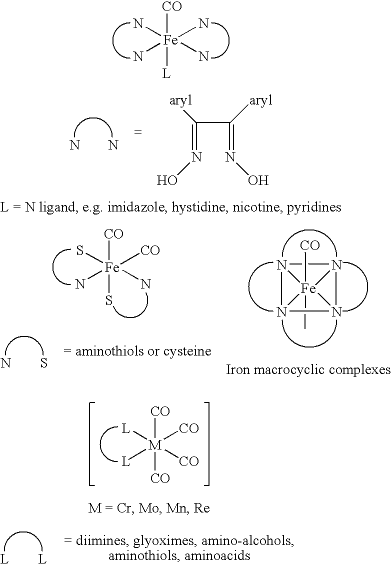

Class 5—CO Containing Inorganic Complexes Bearing Ligands Containing N and/or S Donors that Function as Reversible CO Carriers, Modified by Linkage to Other Pharmacologically Important Molecules.

Following the lines of thought outlined above for Class 2 compounds, new CO carriers of the type described as Class 4, but modified by linking the ligands to other biologically active molecules via an appropriate spacer, are described.

### Examples:

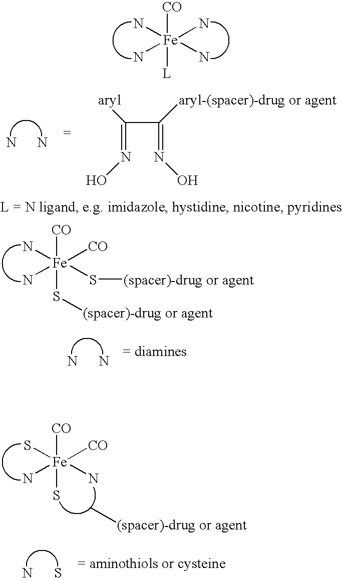

Class 6—Organic Substances that Release CO Either by an Enzymatic Process or by Decarbonylation.

In spite of the fact that decarbonylation is not a very common type of reaction in organic chemistry, some organic substances are known to liberate CO upon treatment with either bases, acids, or radical initiators depending on their nature. These substances fall into the following groups: polyhalomethanes of the general form CHₙX_{y}X′_{4-(n+y)} (X and or X′=F, Cl, Br, I) trichloroacetic acid, and its salts, organic and inorganic esters and sulfinates thereof, triaryl carboxylic acid, formic acid, oxalic acid, α-hydroxyacids and α-ketoacids, esters and salts thereof, under acid conditions; trialkyl and trialkoxybenzaldehydes under acid catalysis; aliphatic aldehydes with radical initiators, e.g., peroxides or light. For the polyhalomethanes, the values of n and y vary in the following way: for n=0, y=1, 2, 3, 4; for n=1, y=1, 2, 3; for n=2, y=1, 2; for n=3, y=1. In the above examples, the term “salt” applies to the ionic derivative of the conjugate base of a given protonic acid, namely a carboxylate, with a main group element ion, namely Na⁺, K⁺. Alkyl is the general name given to the radical of an aliphatic hydrocarbon chain, e.g. methyl, ethyl, propyl, butyl, etc. The alkyl group can be branched or straight chain. Aryl is the general name given to a radical of an aromatic ring, e.g., phenyl, tolyl, xylyl, etc. The aryl group will typically have about 6 to about 10 carbon atoms. Ester is the general name given to the functional group —C(O)OR (where R=alkyl, aryl).

The first two categories produce dichlorocarbene, which, under physiological conditions, will be metabolized to CO. In the case of dichloromethane, cytochrome P-450 has been shown to be responsible for the liberation of CO in vivo.

The third group of compounds releases CO under acid catalysis and is sensitive to the aryl substitution pattern. Most likely this is also true for the fourth group which includes trialkyl and triaryl substituted aldehydes. Strong activating groups on the aryl ring favor CO liberation under acid conditions. More importantly, the radical initiated decomposition of aliphatic aldehydes, induced by peroxides or light, produces CO under very mild conditions. The value of “n”, the number of substituents (alkyl, aryl, alkoxy, aryloxy) on the aromatic ring, can vary from 0 to 5, preferably 1, 2, or 3.

### Examples:

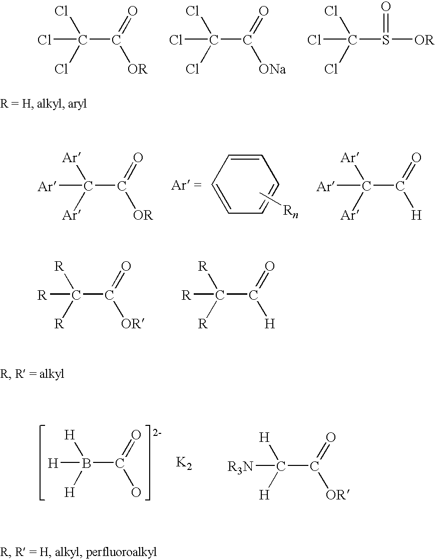

Other examples of CORM aldehydes include compounds of Formula VI:

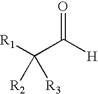

wherein R₁, R₂ and R₃ are each independently selected from H, alkyl, substituted alkyl, cycloalkyl, substituted cycloalkyl, heterocyclyl, substituted heterocyclyl, alkylheterocyclyl, substituted alkylheterocyclyl, alkenyl, substituted alkenyl, aryl, substituted aryl, heteroaryl, substituted heteroaryl, alkylaryl, substituted alkylaryl, hydroxy, alkoxy, amino, alkylamino, mercapto, alkylmercapto, aryloxy, substituted aryloxy, heteroaryloxy, substituted heteroaryloxy, alkoxycarbonyl, acyl, acyloxy, acylamino, alkylsulfonyl, alkylsulfinyl, F, Cl, Br, NO₂ and cyano; or two or more of R₁, R₂ and R₃ are taken together to form a substituted or unsubstituted carbocyclic or heterocyclic ring structure.

“Alkyl” refers to straight or branched chain saturated hydrocarbyl groups having up to 20 carbon atoms, and “substituted alkyl” refers to alkyl groups bearing one or more substituents selected from amino, alkylamino, hydroxy, alkoxy, mercapto, alkylmercapto, aryl, aryloxy, alkoxycarbonyl, acyl, acyloxy, acylamino, F, Cl, Br, NO₂, cyano, sulfonyl, sulfinyl and similar substituents known to those of skill in the art. “Cycloalkyl” refers to saturated hydrocarbyl groups containing one or more rings and having in the range of 3 to 12 carbon atoms, and “substituted cycloalkyl” refers to cycloalkyl groups further bearing one or more substituents as set forth above. “Heterocyclyl” refers to cyclic groups containing one or more rings including one or more heteroatoms (e.g., N, O or S) as part of the ring structure and having in the range of 3 to 12 ring atoms, and “substituted heterocyclyl” refers to heterocyclyl groups further bearing one or more substituents as set forth above. “Alkylheterocyclyl” refers to alkyl-substituted heterocyclyl groups, and “substituted alkylheterocyclyl” refers to alkylheterocyclyl groups further bearing one or more substituents as set forth above. “Alkenyl” refers to straight or branched chain hydrocarbyl groups having at least one carbon-carbon double bond, and having in the range of 2 to 20 carbon atoms, and “substituted alkenyl” refers to alkenyl groups further bearing one or more substituents as set forth above. “Aryl” refers to aromatic groups having in the range of 6 up to about 14 carbon atoms, and “substituted aryl” refers to aryl groups further bearing one or more substituents as set forth above. “Heteroaryl” refers to aromatic groups containing one or more heteroatoms (e.g., N, O or S) as part of the ring structure, and having in the range of 5 up to about 13 carbon atoms, and “substituted heteroaryl” refers to heteroaryl groups further bearing one or more substituents as set forth above. “Alkylaryl” refers to alkyl-substituted aryl groups, and “substituted alkylaryl” refers to alkylaryl groups further bearing one or more substituents as set forth above.

“Hydroxy” refers to the group OH. “Alkoxy” refers to a group —OR, wherein R is an alkyl group as defined above. “Amino” refers to the group NH₂. “Alkylamino” refers to a group —NHR or —NRR′, where R and R′ are independently chosen from alkyl or cycloalkyl groups as defined above. “Mercapto” refers to the group SH. “Alkylmercapto” refers to the group S—R, where R represents an alkyl or cycloalkyl group as defined above. “Aryloxy” refers to a group —OAr, wherein Ar is an aryl group as defined above, and “substituted aryloxy” refers to aryloxy groups further bearing one or more substituents as set forth above. “Heteroaryloxy” refers to a group —OHt, wherein Ht is a heteroaryl group as defined above, and “substituted heteroaryloxy” refers to heteroaryloxy groups further bearing one or more substituents as set forth above. “Alkoxycarbonyl” refers to a group —C(O)—OR, wherein R is an alkyl group as defined above.

“Acyl” refers to a group —C(O)—R, where R is H, alkyl, substituted alkyl, cycloalkyl, substituted cycloalkyl, alkenyl, substituted alkenyl, aryl, substituted aryl, heteroaryl or substituted heteroaryl, as defined above. “Acyloxy” refers to a group —O—C(O)—R, where R is H, alkyl, substituted alkyl, cycloalkyl, substituted cycloalkyl, alkenyl, substituted alkenyl, aryl, substituted aryl, heteroaryl or substituted heteroaryl, as defined above. “Acylamino” refers to a group —NR′C(O)R, where R and R′ are each independently chosen from H, alkyl, substituted alkyl, cycloalkyl, substituted cycloalkyl, alkenyl, substituted alkenyl, aryl, substituted aryl, heteroaryl or substituted heteroaryl, as defined above. “Alkylsulfonyl” refers to a group —S(O)₂R, where R represents an alkyl or cycloalkyl group as defined above. “Alkylsulfinyl” refers to a group —S(O)R, where R represents an alkyl or cycloalkyl group as defined above.

Non-limiting examples of aldehydes of the general Formula VI include the following:

trimethylacetaldehyde (compound 1)

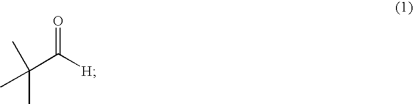

2,2-dimethyl-4-pentenal (compound 2)

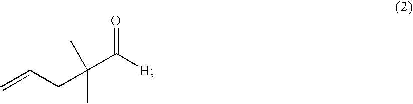

4-ethyl-4-formyl-hexanenitrile (compound 3)

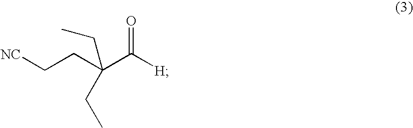

3-hydroxy-2,2-dimethylpropanal (compound 4)

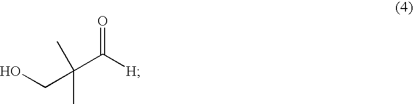

2-formyl-2-methyl-propylmethanoate (compound 5)

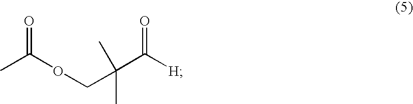

2,2-dimethyl-3-(p-methylphenyl)propanal (compound 6)

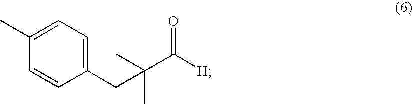

2-methyl-2-phenylpropionaldehyde (compound 7)

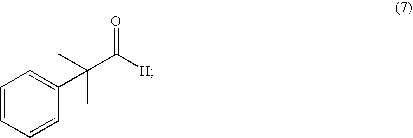

and 2-ethyl-2-methyl-propionaldehyde (compound 8)

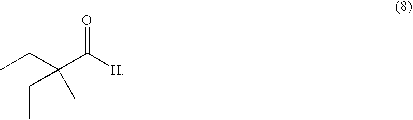

The most common reactions known for the decarbonylation of aldehydes require drastic conditions, such as strong acidic or basic conditions, high temperatures together with ultraviolet light, radical initiators and/or the presence of a metal catalyst (Jerry March, Advanced Organic Chemistry, Reactions, Mechanisms and Structure, John Wiley & Sons, 4^{th} Ed., 1992). However, highly branched aldehydes have been observed to decarbonylate at room temperature when irradiated by ultraviolet light (Berman et al., J. Am. Chem. Soc., 85:4010-4013 (1963); Conant et al., J. Am. Chem. Soc. 51:1246-1255 (1929)). The loss of carbon monoxide from tertiary aldehydes leads to tertiary radicals, which are more stable than primary or secondary radicals due to resonance stabilization by hyperconjugation. Hyperconjugation includes the stabilization that results from the interaction of electrons in a σ-bond (usually C—H or C—C) with an adjacent empty (or partially filled) p-orbital or π-orbital to give an extended molecular orbital that increases the stability of the system. Thus, decarbonylation is favored in tertiary aldehydes, as compared to primary and secondary aldehydes.

While not to be bound by any particular theory, the following equation 1 shows a proposed mechanism for the decarbonylation of tertiary aldehydes (exemplified by trimethylacetaldehyde (compound 1)) by reactive oxygen species, generating carbon monoxide and a stabilized tertiary radical:

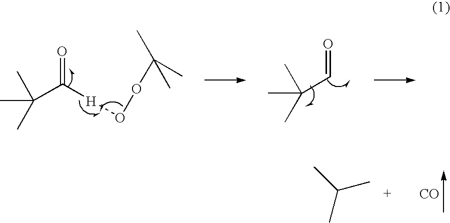

Accordingly, in certain embodiments, the aldehyde is a tertiary aldehyde. In such embodiments, the aldehyde is a compound of the above Formula VI in which R₁, R₂ and R₃ are each independently selected from alkyl, substituted alkyl, cycloalkyl, substituted cycloalkyl, heterocyclyl, substituted heterocyclyl, alkylheterocyclyl, substituted alkylheterocyclyl, alkenyl, substituted alkenyl, aryl, substituted aryl, heteroaryl, substituted heteroaryl, alkylaryl, substituted alkylaryl, hydroxy, alkoxy, amino, alkylamino, mercapto, alkylmercapto, aryloxy, substituted aryloxy, heteroaryloxy, substituted heteroaryloxy, alkoxycarbonyl, acyl, acyloxy, acylamino, alkylsulfonyl, alkylsulfinyl, F, Cl, Br, NO₂ and cyano; or two or more of R₁, R₂ and R₃ are taken together to form a substituted or unsubstituted carbocyclic or heterocyclic ring structure.

In some instances, for example, to improve the in vivo stability, bioavailability, or pharmacokinetic properties of a therapeutic aldehyde, the aldehyde is administered in the form of a derivative, or a protected form thereof. The derivative serves as a source of the free or unmodified aldehyde in vivo and/or releases CO in vivo itself. In certain embodiments, an aldehyde derivative is generated that acts as a prodrug, a pharmacologically inactive chemical entity that, when chemically transformed or metabolised in an animal, is converted into a pharmacologically active substance. The generation of the therapeutically effective molecule (i.e., the aldehyde) from the prodrug occurs prior to, during or after reaching the site of action within the body (Bundgaard et al., Int. J. Pharm. 13:89-98 (1983)). Release of the aldehyde from the prodrug generally occurs via chemical or enzymatic lability, or both, within the body system.

Examples of aldehyde prodrugs that are chemically labile include, without limitation, non-cyclic chain compounds that exist in equilibrium in physiological media, such as Mannich base derivatives, imines, oximes, amidines, hydrazones and semicarbazones (WO 2006/012215; Herrmann et al., Chem. Commun. 2965-2967 (2006); Deaton et al., Bioorg. Med. Chem. Lett. 16:978-983 (2006)), and ring chain tautomeric prodrugs such as 1,3-X,N-heterocycles (X═O, S, NR) (Valters et al., Adv. Heterocycl. Chem. 64:251-321 (1995); Valters et al., Adv. Heterocycl. Chem. 66:1-71 (1996)) that are prepared from the reaction of difunctional compounds with aldehydes. From the ring chain equilibria of these derivatives, the open form undergoes hydrolysis to give the bioactive molecule. In both cases, the ratios of the species involved in the equilibria of these systems are strongly influenced by the steric and electronic characters of the substituents.

An alternative strategy is to generate prodrugs that are converted to the pharmacologically active compound by an enzymatic process (Bernard Testa & Joachim M. Mayer, Hydrolysis in Drug and Prodrug Metabolism, Chemistry, Biochemistry and Enzymology WILEY-VCH, 2003). There are several types of chemical groups such as, for example, esters, amides, sulphates and phosphates, that are readily cleaved by esterases, aminases, sulphatases and phosphatases, respectively. Pharmacologically active aldehydes are released by the action of esterases and amidases on a variety of compounds that include acyloxyalkyl esters, N-acyloxyalkyl derivatives, N-Mannich bases derivative, N-hydroxymethyl derivatives, and others. In some instances, to facilitate hydrolysis when the prodrug is a poor substrate for the aldehyde-generating enzyme, the carrier is modified with electron withdrawing or donating groups.

As recognized by those skilled in the art, organic aldehydes undergo a variety of reactions that render the aldehyde chemically protected. By way of non-limiting example, in various embodiments, organic aldehydes are protected by conversion to the corresponding acetal, hemiacetal, aminocarbinol, aminal, imine, enaminone, imidate, amidine, iminium salt, sodium bissulfite adduct, hemimercaptal, dithioacetal, 1,3-dioxepane, 1,3-dioxane, 1,3-dioxalane, 1,3-dioxetane, α-hydroxy-1,3-dioxepane, α-hydroxy-1,3-dioxane, α-hydroxy-1,3-dioxalane, α-keto-1,3-dioxepane, α-keto-1,3-dioxane, α-keto-1,3-dioxalane, α-keto-1,3-dioxetane, macrocyclic ester/imine, macrocyclic ester/hemiacetal, oxazolidine, tetrahydro-1,3-oxazine, oxazolidinone, tetrahydro-oxazinone, 1,3,4-oxadiazine, thiazolidine, tetrahydro-1,3-thiazine, thiazolidinone, tetrahydro-1,3-thiazinone, imidazolidine, hexahydro-1,3-pyrimidine, imidazolidinone, tetrahydro-1,3-pyrimidinone, oxime, hydrazone, carbazone, thiocarbazone, semicarbazone, semithiocarbazone, acyloxyalkyl ester derivative, O-acyloxyalkyl derivative, N-acyloxyalkyl derivative, N-Mannich base derivative, or N-hydroxymethyl derivative. The exemplary schemes in equations 2-12 below also illustrate how many such prodrugs release the active aldehyde in vivo (e.g., via hydrolytic or enzymatic hydrolysis).

In certain embodiments, the protected organic aldehyde is an imine. Those skilled in the art recognize that such derivatives are obtained in a variety of ways, such as, for example, by the methods described by Deaton et al., Bioorg. Med. Chem. Lett. 16: 978-983 (2006), or WO2006/012215, by reaction of an organic aldehyde with an amine as in equation 2:

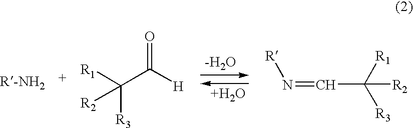

wherein each of R₁, R₂ and R₃ is independently selected from H, alkyl, substituted alkyl, cycloalkyl, substituted cycloalkyl, heterocyclyl, substituted heterocyclyl, alkylheterocyclyl, substituted alkylheterocyclyl, alkenyl, substituted alkenyl, aryl, substituted aryl, heteroaryl, substituted heteroaryl, alkylaryl, substituted alkylaryl, hydroxy, alkoxy, amino, alkylamino, mercapto, alkylmercapto, aryloxy, substituted aryloxy, heteroaryloxy, substituted heteroaryloxy, alkoxycarbonyl, acyl, acyloxy, acylamino, alkylsulfonyl, alkylsulfinyl, F, Cl, Br, NO₂ and cyano; or two or more of R₂ and R₃ are taken together to form a substituted or unsubstituted carbocyclic or heterocyclic ring structure; and

R′ is selected from H, alkyl, substituted alkyl, cycloalkyl, substituted cycloalkyl, alkenyl, substituted alkenyl, aryl, substituted aryl, heteroaryl and substituted heteroaryl.

In other embodiments, the protected organic aldehyde is an iminium salt. Those skilled in the art recognize that such derivatives can be obtained in a variety of ways, such as, for example, by the methods described by Paukstelis et al., J. Org. Chem. 28:3021-3024 (1963), by reaction of an organic aldehyde with a secondary amine salt as in equation 3:

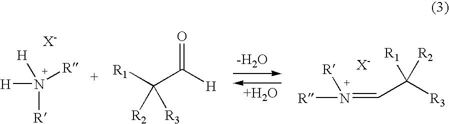

wherein each of R₁, R₂, R₃ and R′ is as defined above with respect to equation 2;

R″ is selected from H, alkyl, substituted alkyl, cycloalkyl, substituted cycloalkyl, alkenyl, substituted alkenyl, aryl, substituted aryl, heteroaryl and substituted heteroaryl;

and X represents any suitable and pharmaceutically acceptable counter anion, such as chloride, bromide, phosphate, carbonate, sulfate, acetate or any other non-toxic, physiologically compatible anion.

In another embodiment, the protected organic aldehyde is a hydrazone. Those skilled in the art recognize that such derivatives are prepared in a number of ways such as, for example, by the methods disclosed in U.S. Pat. Nos. 6,518,269 and 4,983,755, by reaction of an organic aldehyde with a hydrazine as in equation 4:

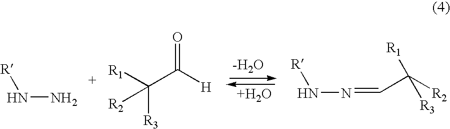

wherein each of R₁, R₂, R₃ and R′ is as defined above with respect to equation 2.

In yet another embodiment, the protected organic aldehyde is a carbazone. Those skilled in the art recognize that such derivatives can be obtained in a variety of ways such as, for example, using methods described by Herrmann et al., Chem. Commun. 2965-2967 (2006) by reaction of an organic aldehyde with a hydrazide (or acyl hydrazine) as in equation 5:

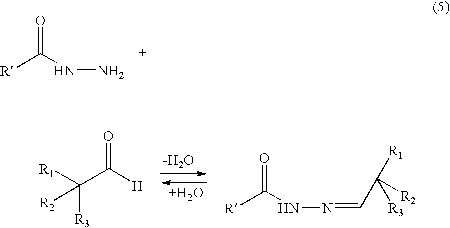

wherein each of R₁, R₂, R₃ and R′ is as defined above with respect to equation 2.

In another embodiment, the protected organic aldehyde is a semicarbazone or thiosemicarbazone. Those skilled in the art recognize that such derivatives can be obtained in a variety of ways, such as, for example, using the methods described by Deaton et al., Bioorg. Med. Chem. Lett. 16:978-983 (2006) or by the methods disclosed in U.S. Pat. No. 6,458,843, for example, by reaction of an organic aldehyde with a semicarbazine or thiosemicarbazine as in equation 6:

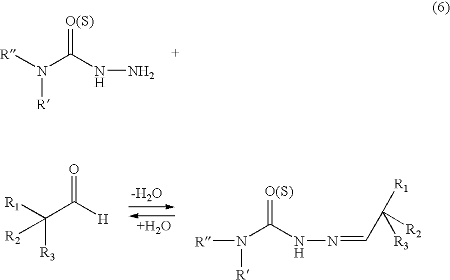

wherein each of R₁, R₂, R₃, R″ is as defined above with respect to equations 2 and 3.

In still another embodiment, the protected organic aldehyde is an oxime. Those skilled in the art recognize that such derivatives can be obtained in a variety of ways, such as, for example, using the methods described by Reymond et al., Org. Biomol. Chem. 2:1471-1475 (2004) or U.S. Patent Application No. 2006/0058513, by reaction of an organic aldehyde with an oxoamine as in equation 7:

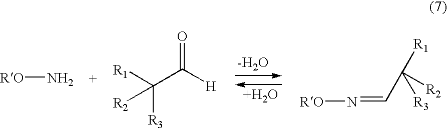

wherein each of R₁, R₂, R₃ and R′ is as defined above with respect to equation 2.

In another embodiment, the protected organic aldehyde is an acetal or hemiacetal. Those skilled in the art recognize that such derivatives can be prepared in a variety of ways, such as, for example, by reaction of an aldehyde with one or more alcohols as in equation 8:

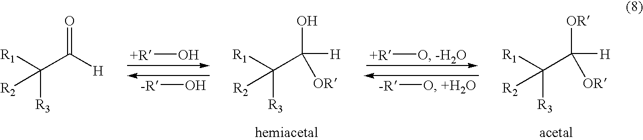

wherein each of R₁, R₂, R₃ and R′ is as defined above with respect to equation 2.

In still another embodiment, the protected organic aldehyde is an α-hydroxy-1,3-dioxepane (or α-hydroxy-1,3-dioxane or α-hydroxy-1,3-dioxalane). Those skilled in the art recognize that such derivatives can be obtained in a variety of ways, such as, for example, by the methods disclosed in WO03/082850, by reaction of a hydroxy substituted organic aldehyde with another aldehyde, as in equation 9:

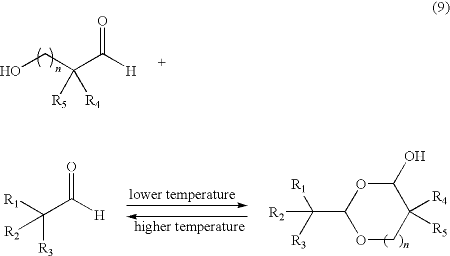

wherein each of R₁, R₂ and R₃ is as defined above with respect to equation 2; each of R₄ and R₅ is independently selected from H, alkyl, substituted alkyl, cycloalkyl, substituted cycloalkyl, heterocyclyl, substituted heterocyclyl, alkylheterocyclyl, substituted alkylheterocyclyl, alkenyl, substituted alkenyl, aryl, substituted aryl, heteroaryl, substituted heteroaryl, alkylaryl, substituted alkylaryl, hydroxy, alkoxy, amino, alkylamino, mercapto, alkylmercapto, aryloxy, substituted aryloxy, heteroaryloxy, substituted heteroaryloxy, alkoxycarbonyl, acyl, acyloxy, acylamino, alkylsulfonyl, alkylsulfinyl, F, Cl, Br, NO₂ and cyano; or R₄ and R₅ are taken together to form a substituted or unsubstituted carbocyclic or heterocyclic ring structure; and n is 1, 2 or 3.

The reaction shown in equation 9 is an energetically favorable cyclization (dimerization) that occurs spontaneously when the compounds are cooled together (1:1) to room temperature. When heated (e.g., to physiological temperatures), they separate again. Compound 4 is an example of a compound that forms a dimer upon cooling to room temperature.

In yet another embodiment, the protected organic aldehyde is an α-keto-1,3-dioxepane (or α-keto-1,3-dioxane, α-keto-1,3-dioxalane or α-keto-1,3-dioxetane). Those skilled in the art recognize that such derivatives can be obtained in a variety of ways, such as, for example, by the methods described by Xu et al., Tet. Lett., 46:3815-3818 (2005) or Krall et al., Tetrahedron 61:137-143 (2005), by reaction of an organic aldehyde with a hydroxy acid, thereby forming a protected aldehyde, as in equation 10:

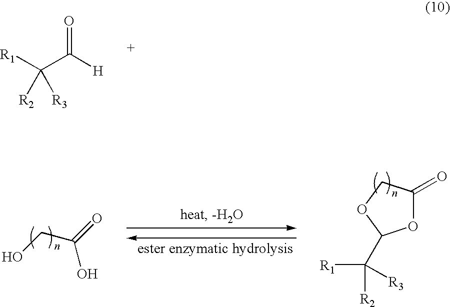

wherein each of R₁, R₂ and R₃ is as defined above with respect to equation 2; and n is 0, 1, 2, or 3.

In another embodiment, the protected organic aldehyde is a macrocyclic ester/imine. Those skilled in the art recognize that such derivatives can be obtained in a variety of ways, such as, for example, as described in U.S. Pat. No. 6,251,927, by reaction of a hydroxy substituted organic aldehyde with a compound of the formula HOOC—(CH₂)ₘ—NH₂, thereby forming a protected aldehyde, as in equation 11:

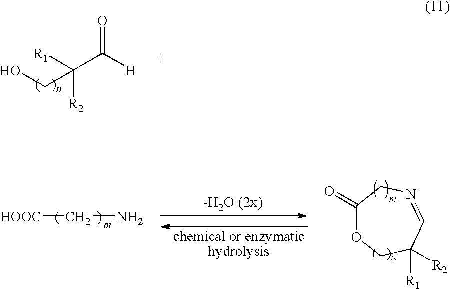

wherein R₁ and R₂ are as defined above with respect to equation 2; n is 0, 1, or 2; and m is 1 or 2.

Hydrolysis of the compound formed in equation 11 occurs by chemical hydrolysis through the imine, or enzymatic hydrolysis through the ester group.

In another embodiment, the protected organic aldehyde is a macrocyclic ester/hemiacetal. Those skilled in the art recognize that such derivatives can be obtained in a variety of ways, such as, for example, as described in U.S. Pat. No. 6,251,927 by reaction of a hydroxy substituted organic aldehyde with a hydroxy acid having the structure HOOC—(CH₂)ₘ—OH, thereby forming a protected aldehyde, as in equation 12: HOOC—(CH₂)ₘ—OH, thereby forming a protected aldehyde, as in equation 12:

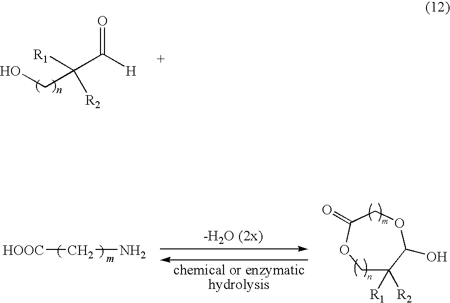

wherein R₁, R₂, in and n are as defined above with respect to equation 11.

Hydrolysis of the compound formed in equation 12 occurs by chemical hydrolysis through the ketal, or enzymatic hydrolysis through the ester group.

In still another embodiment, the protected organic aldehyde is a thiazolidine or a tetrahydro-1,3-thiazine. Those skilled in the art recognize that such derivatives can be obtained in a variety of ways, such as, for example, by employing the methods described by Jellum et al., Anal. Biochem. 31:339-347 (1969), Nagasawa et al., J. Biochem. Mol. Tox. 16:235-244 (2002), Roberts et al., Chem. Res. Toxicol. 11:1274-82 (1998) or U.S. Pat. No. 5,385,922. Certain thiazolidines and tetrahydro-1,3-thiazines contemplated for use as described herein are represented by Formula VII:

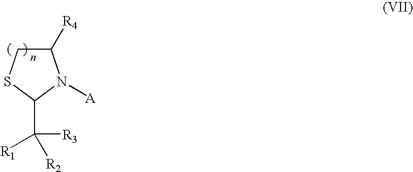

wherein each of R₁, R₂, R₃ and R₄ is independently selected from H, alkyl, substituted alkyl, cycloalkyl, substituted cycloalkyl, heterocyclyl, substituted heterocyclyl, alkylheterocyclyl, substituted alkylheterocyclyl, alkenyl, substituted alkenyl, aryl, substituted aryl, heteroaryl, substituted heteroaryl, alkylaryl, substituted alkylaryl, hydroxy, alkoxy, amino, alkylamino, mercapto, alkylmercapto, aryloxy, substituted aryloxy, heteroaryloxy, substituted heteroaryloxy, alkoxycarbonyl, acyl, acyloxy, acylamino, alkylsulfonyl, alkylsulfinyl, F, Cl, Br, NO₂, and cyano; or two or more of R₁, R₂ and R₃ are taken together to form a substituted or unsubstituted carbocyclic or heterocyclic ring structure;

A is selected from H, alkyl, substituted alkyl, cycloalkyl, substituted cycloalkyl, alkenyl, substituted alkenyl, aryl, substituted aryl, heteroaryl, substituted heteroaryl, alkylaryl, substituted alkylaryl, alkoxycarbonyl, acyl, acyloxy, acylamino, alkylsulfonyl and alkylsulfinyl; and n is 1 or 2.

In another embodiment, the protected organic aldehyde is an oxazolidine or a tetrahydro-1,3-oxazine. Those skilled in the art recognize that such derivatives can be obtained in a variety of ways, such as, for example, by employing the methods described by Bundgaard et al., Int. J. Pharma. Chem. 10:165-175 (1982), Sélambarom et al., Tetrahedron 58:9559-9556 (2002) or U.S. Pat. No. 7,018,978. Certain oxazolidines and tetrahydro-1,3-oxazines contemplated for use as described herein are represented by Formula VIII:

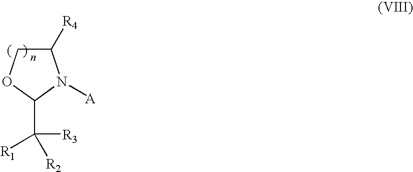

wherein each of R₁, R₂, R₃, R₄ and A and n is as described above with respect to formula VII.

In still another embodiment, the protected organic aldehyde is an imidazolidine or a 1,3-hexahydro-pyrimidine. Those skilled in the art recognize that such derivatives can be obtained in a variety of ways, such as, for example, by employing the methods described by Lambert, J. Org. Chem. 52:68-71 (1987) or Mop, J. Org. Chem. 67:4734-4741 (2002). Certain imidazolidines and 1,3-hexahydro-pyrimidines contemplated for use as described herein are represented by Formula IX:

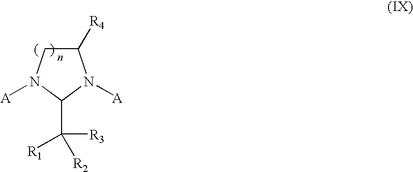

wherein each of R₁, R₂, R₃, R₄, n and A (selected independently at each occurrence) is as described above with respect to Formula VII.

In yet another embodiment, the protected organic aldehyde is an imidazolidinone. Those skilled in the art recognize that such derivatives can be obtained in a variety of ways, such as, for example, by employing the methods described by Bundgaard et al., Int. J. Pharma. Chem. 23:163-173 (1985). Certain imidazolidinones contemplated for use as described herein are represented by Formula X:

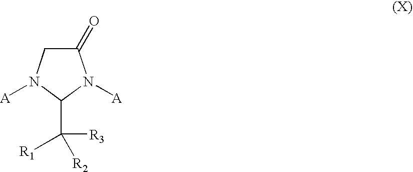

wherein each of R₁, R₂, R₃ and A (selected independently at each occurrence) is as described above with respect to Formula VII.

In another embodiment, the protected organic aldehyde is an acyloxyalkyl ester or O-acyloxyalkyl derivative. Those skilled in the art recognize that such derivatives can be obtained in a variety of ways, such as, for example, by employing the methods described by Nudelman et al., Eur J. Med. J. Chem. 36: 63-74 (2001), Nudelman et al., J. Med. Chem. 48:1042-1054 (2005), or Swedish Patent No. SE9301115. Certain acyloxyalkyl esters contemplated for use as described herein are represented by Formula XI:

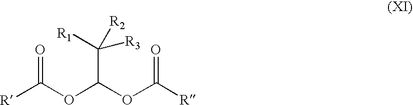

wherein each of R₁, R₂, and R₃ is as defined above with respect to Formula VII, and each of R′ and R″ is selected independently from H, alkyl, substituted alkyl, cycloalkyl, substituted cycloalkyl, alkenyl, substituted alkenyl, aryl, substituted aryl, heteroaryl, and substituted heteroaryl. In certain embodiments, in addition to releasing the active aldehyde upon metabolic hydrolysis in vivo, an acyloxyalkyl ester derivative also releases butyric acid. Butyric acid prodrugs have been reported to provide increased aqueous solubility and permeability across cell membranes (Nudelman et al., Eur J. Med. J. Chem. 36: 63-74 (2001)).

In another embodiment, the protected organic aldehyde is an N-acyloxyalkyl derivative. Those skilled in the art recognize that such derivatives can be obtained in a variety of ways, such as, for example, by employing the methods described by Bundgaard et al., Int. J. Pharm. 22:454-456 (1984) and Bundgaard et al., Int. J. Pharm. 13:89-98 (1983). Certain N-acyloxyalkyl derivatives contemplated for use as described herein are represented by Formula XII:

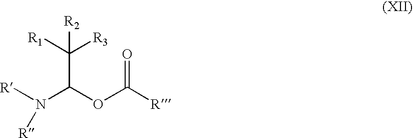

wherein each of R₁, R₂, R₃, R′ and R″ is as described above with respect to Formulas VII and IX; and R″′ is selected from H, alkyl, substituted alkyl, cycloalkyl, substituted cycloalkyl, alkenyl, substituted alkenyl, aryl, substituted aryl, heteroaryl, and substituted heteroaryl.

In another embodiment, the protected organic aldehyde is the salt of an N-acyloxyalkyl derivative. Those skilled in the art recognize that such derivatives can be obtained in a variety of ways, such as, for example, by employing the methods described by Bodor et al., J. Med. Chem. 23:469-474 (1980) or U.S. Pat. No. 3,998,815. The salts of N-acyloxyalkyl derivatives contemplated for use as described herein are represented by Formula XIII:

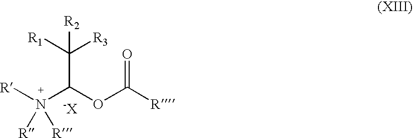

wherein each of R₁, R₂, R₃, R′, R″, and R″′ is as defined above with respect to formula X;

R″″ is selected from H, alkyl, substituted alkyl, cycloalkyl, substituted cycloalkyl, alkenyl, substituted alkenyl, aryl, substituted aryl, heteroaryl, and substituted heteroaryl; and

X represents a suitable and pharmaceutically acceptable counter anion, as described above with respect to equation 3.

In yet another embodiment, the protected organic aldehyde is a 5-oxazolidinone.

Those skilled in the art recognize that such derivatives can be obtained in a variety of ways, such as, for example, by employing the methods described by Bundgaard et al., Int. J. Pharma. Chem. 46:159-167 (1988) or Ishai-Ben, J. Am. Chem. Soc. 79:5736-38 (1957). Certain 5-oxazolidinones contemplated for use as described herein are represented by Formula XIV:

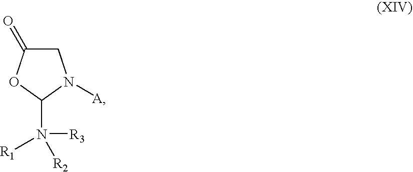

wherein each of R₁, R₂, R₃ and A is as defined above with respect to Formula VII.
Class 7—Encapsulated Organic Substances that Release CO Either by an Enzymatic Process or by Decarbonylation.

This system comprises the same molecules described under Class 6, but includes their encapsulation in host-guest supermolecules, liposomes, cyclodextrins, and other polymeric materials that are able to produce nanoencapsulated drug delivery products.

Other sources of CO include: tricarbonyldichlororuthenium (II) dimmer, CORM-2 (Sigma); tricarbonylchloro(glycinato)ruthenium (II), CORM-3 (Johnson, T. R. et al. Dalton Trans, 1500-8 (2007).); bromo(pentacarbonyl)manganese, (Herrmann-Brauer. Synthetic Methods of Organometallic and Inorganic Chemistry (ed. Herrmann, W. A.) (Stuttgart, 1997).) and tetraethylammonium molybdenum pentacarbonyl bromide (Burgmayer, S. J. N. & L., T. J. Inorganic Chemistry 24, 2224-2230 (1985).)

The CO may be administered alone, in a pharmaceutical composition or combined with other therapeutic regimens. The CO and other therapeutic agent(s) may be administered simultaneously or sequentially. When the other therapeutic agents are administered simultaneously they can be administered in the same or separate formulations, but are administered at the same time. The other therapeutic agents may be administered sequentially with one another and with CO when the administration of the other therapeutic agents and the CO is temporally separated. The separation in time between the administration of these compounds may be a matter of minutes or it may be longer. Other therapeutic agents include but are not limited to anti-infective agent(s). Examples of anti-infective agent(s) include: anti-bacterial agent(s), anti-viral agent(s), anti-fungal agent(s) or anti-protozoal agent(s).

Phrases such as “anti-infective agent”, “anti-bacterial agent”, “anti-viral agent”, “anti-fungal agent”, “anti-parasitic agent” and “parasiticide” have well-established meanings to those of ordinary skill in the art and are defined in standard medical texts. Briefly, anti-bacterial agents kill or inhibit the growth or function of bacteria. Anti-bacterial agents include antibiotics as well as other synthetic or natural compounds having similar functions. Antibiotics, typically, are low molecular weight molecules which are produced as secondary metabolites by cells, such as microorganisms. In general, antibiotics interfere with one or more bacterial functions or structures which are specific for the microorganism and which are not present in host cells.

A large class of anti-bacterial agents is antibiotics. Antibiotics that are effective for killing or inhibiting a wide range of bacteria are referred to as broad spectrum antibiotics. Other types of antibiotics are predominantly effective against the bacteria of the class gram-positive or gram-negative. These types of antibiotics are referred to as narrow spectrum antibiotics. Other antibiotics which are effective against a single organism or disease and not against other types of bacteria, are referred to as limited spectrum antibiotics. Anti-bacterial agents are sometimes classified based on their primary mode of action. In general, anti-bacterial agents are cell wall synthesis inhibitors, cell membrane inhibitors, protein synthesis inhibitors, nucleic acid synthesis or functional inhibitors, and competitive inhibitors.

Anti-bacterial agents include but are not limited to aminoglycosides, β-lactam agents, cephalosporins, macrolides, penicillins, quinolones, sulfonamides, and tetracyclines. Examples of anti-bacterial agents include but are not limited to: Acedapsone, Acetosulfone Sodium, Alamecin, Alexidine, Amdinocillin Clavulanate Potassium, Amdinocillin, Amdinocillin Pivoxil, Amicycline, Amifloxacin, Amifloxacin Mesylate, Amikacin, Amikacin Sulfate, Aminosalicylic acid, Aminosalicylate sodium, Amoxicillin, Amphomycin, Ampicillin, Ampicillin Sodium, Apalcillin Sodium, Apramycin, Aspartocin, Astromicin Sulfate, Avilamycin, Avoparcin, Azithromycin, Azlocillin, Azlocillin Sodium, Bacampicillin Hydrochloride, Bacitracin, Bacitracin Methylene Disalicylate, Bacitracin Zinc, Bambermycins, Benzoylpas Calcium, Berythromycin, Betamicin Sulfate, Biapenem, Biniramycin, Biphenamine Hydrochloride, Bispyrithione Magsulfex, Butikacin, Butirosin Sulfate, Capreomycin Sulfate, Carbadox, Carbenicillin Disodium, Carbenicillin Indanyl Sodium, Carbenicillin Phenyl Sodium, Carbenicillin Potassium, Carumonam Sodium, Cefaclor, Cefadroxil, Cefamandole, Cefamandole Nafate, Cefamandole Sodium, Cefaparole, Cefatrizine, Cefazaflur Sodium, Cefazolin, Cefazolin Sodium, Cefbuperazone, Cefdinir, Cefditoren Pivoxil, Cefepime, Cefepime Hydrochloride, Cefetecol, Cefixime, Cefmenoxime Hydrochloride, Cefmetazole, Cefmetazole Sodium, Cefonicid Monosodium, Cefonicid Sodium, Cefoperazone Sodium, Ceforanide, Cefotaxime, Cefotaxime Sodium, Cefotetan, Cefotetan Disodium, Cefotiam Hydrochloride, Cefoxitin, Cefoxitin Sodium, Cefpimizole, Cefpimizole Sodium, Cefpiramide, Cefpiramide Sodium, Cefpirome Sulfate, Cefpodoxime Proxetil, Cefprozil, Cefroxadine, Cefsulodin Sodium, Ceftazidime, Ceftazidime Sodium, Ceftibuten, Ceftizoxime Sodium, Ceftriaxone Sodium, Cefuroxime, Cefuroxime Axetil, Cefuroxime Pivoxetil, Cefuroxime Sodium, Cephacetrile Sodium, Cephalexin, Cephalexin Hydrochloride, Cephaloglycin, Cephaloridine, Cephalothin Sodium, Cephapirin Sodium, Cephradine, Cetocycline Hydrochloride, Cetophenicol, Chloramphenicol, Chloramphenicol Palmitate, Chloramphenicol Pantothenate Complex, Chloramphenicol Sodium Succinate, Chlorhexidine Phosphanilate, Chloroxylenol, Chlortetracycline Bisulfate, Chlortetracycline Hydrochloride, Cilastatin, Cinoxacin, Ciprofloxacin, Ciprofloxacin Hydrochloride, Cirolemycin, Clarithromycin, Clavulanate Potassium, Clinafloxacin Hydrochloride, Clindamycin, Clindamycin Dextrose, Clindamycin Hydrochloride, Clindamycin Palmitate Hydrochloride, Clindamycin Phosphate, Clofazimine, Cloxacillin Benzathine, Cloxacillin Sodium, Cloxyquin, Colistimethate, Colistimethate Sodium, Colistin Sulfate, Coumermycin, Coumermycin Sodium, Cyclacillin, Cycloserine, Dalfopristin, Dapsone, Daptomycin, Demeclocycline, Demeclocycline Hydrochloride, Demecycline, Denofungin, Diaveridine, Dicloxacillin, Dicloxacillin Sodium, Dihydrostreptomycin Sulfate, Dipyrithione, Dirithromycin, Doxycycline, Doxycycline Calcium, Doxycycline Fosfatex, Doxycycline Hyclate, Doxycycline Monohydrate, Droxacin Sodium, Enoxacin, Epicillin, Epitetracycline Hydrochloride, Ertapenem, Erythromycin, Erythromycin Acistrate, Erythromycin Estolate, Erythromycin Ethylsuccinate, Erythromycin Gluceptate, Erythromycin Lactobionate, Erythromycin Propionate, Erythromycin Stearate, Ethambutol Hydrochloride, Ethionamide, Fleroxacin, Floxacillin, Fludalanine, Flumequine, Fosfomycin, Fosfomycin Tromethamine, Fumoxicillin, Furazolium Chloride, Furazolium Tartrate, Fusidate Sodium, Fusidic Acid, Gatifloxacin, Genifloxacin, Gentamicin Sulfate, Gloximonam, Gramicidin, Haloprogin, Hetacillin, Hetacillin Potassium, Hexedine, Ibafloxacin, Imipenem, Isoconazole, Isepamicin, Isoniazid, Josamycin, Kanamycin Sulfate, Kitasamycin, Levofloxacin, Levofuraltadone, Levopropylcillin Potassium, Lexithromycin, Lincomycin, Lincomycin Hydrochloride, Linezolid, Lomefloxacin, Lomefloxacin Hydrochloride, Lomefloxacin Mesylate, Loracarbef, Mafenide, Meclocycline, Meclocycline Sulfosalicylate, Megalomicin Potassium Phosphate, Mequidox, Meropenem, Methacycline, Methacycline Hydrochloride, Methenamine, Methenamine Hippurate, Methenamine Mandelate, Methicillin Sodium, Metioprim, Metronidazole Hydrochloride, Metronidazole Phosphate, Mezlocillin, Mezlocillin Sodium, Minocycline, Minocycline Hydrochloride, Mirincamycin Hydrochloride, Monensin, Monensin Sodium, Moxifloxacin Hydrochloride, Nafcillin Sodium, Nalidixate Sodium, Nalidixic Acid, Natamycin, Nebramycin, Neomycin Palmitate, Neomycin Sulfate, Neomycin Undecylenate, Netilmicin Sulfate, Neutramycin, Nifuradene, Nifuraldezone, Nifuratel, Nifuratrone, Nifurdazil, Nifurimide, Nifurpirinol, Nifurquinazol, Nifurthiazole, Nitrocycline, Nitrofurantoin, Nitromide, Norfloxacin, Novobiocin Sodium, Ofloxacin, Ormetoprim, Oxacillin Sodium, Oximonam, Oximonam Sodium, Oxolinic Acid, Oxytetracycline, Oxytetracycline Calcium, Oxytetracycline Hydrochloride, Paldimycin, Parachlorophenol, Paulomycin, Pefloxacin, Pefloxacin Mesylate, Penamecillin, Penicillin G Benzathine, Penicillin G Potassium, Penicillin G Procaine, Penicillin G Sodium, Penicillin V, Penicillin V Benzathine, Penicillin V Hydrabamine, Penicillin V Potassium, Pentizidone Sodium, Phenyl Aminosalicylate, Piperacillin, Piperacillin Sodium, Pirbenicillin Sodium, Piridicillin Sodium, Pirlimycin Hydrochloride, Pivampicillin Hydrochloride, Pivampicillin Pamoate, Pivampicillin Probenate, Polymyxin B Sulfate, Porfiromycin, Propikacin, Pyrazinamide, Pyrithione Zinc, Quindecamine Acetate, Quinupristin, Racephenicol, Ramoplanin, Ranimycin, Relomycin, Repromicin, Rifabutin, Rifametane, Rifamexil, Rifamide, Rifampin, Rifapentine, Rifaximin, Rolitetracycline, Rolitetracycline Nitrate, Rosaramicin, Rosaramicin Butyrate, Rosaramicin Propionate, Rosaramicin Sodium Phosphate, Rosaramicin Stearate, Rosoxacin, Roxarsone, Roxithromycin, Sancycline, Sanfetrinem Sodium, Sarmoxicillin, Sarpicillin, Scopafungin, Sisomicin, Sisomicin Sulfate, Sparfloxacin, Spectinomycin Hydrochloride, Spiramycin, Stallimycin Hydrochloride, Steffimycin, Sterile Ticarcillin Disodium, Streptomycin Sulfate, Streptonicozid, Sulbactam Sodium, Sulfabenz, Sulfabenzamide, Sulfacetamide, Sulfacetamide Sodium, Sulfacytine, Sulfadiazine, Sulfadiazine Sodium, Sulfadoxine, Sulfalene, Sulfamerazine, Sulfameter, Sulfamethazine, Sulfamethizole, Sulfamethoxazole, Sulfamonomethoxine, Sulfamoxole, Sulfanilate Zinc, Sulfanitran, Sulfasalazine, Sulfasomizole, Sulfathiazole, Sulfazamet, Sulfisoxazole, Sulfisoxazole Acetyl, Sulfisoxazole Diolamine, Sulfomyxin, Sulopenem, Sultamicillin, Suncillin Sodium, Talampicillin Hydrochloride, Tazobactam, Teicoplanin, Temafloxacin Hydrochloride, Temocillin, Tetracycline, Tetracycline Hydrochloride, Tetracycline Phosphate Complex, Tetroxoprim, Thiamphenicol, Thiphencillin Potassium, Ticarcillin Cresyl Sodium, Ticarcillin Disodium, Ticarcillin Monosodium, Ticlatone, Tiodonium Chloride, Tobramycin, Tobramycin Sulfate, Tosufloxacin, Trimethoprim, Trimethoprim Sulfate, Trisulfapyrimidines, Troleandomycin, Trospectomycin Sulfate, Trovafloxacin, Tyrothricin, Vancomycin, Vancomycin Hydrochloride, Virginiamycin, Zorbamycin.

Anti-viral agents can be isolated from natural sources or synthesized and are useful for killing or inhibiting the growth or function of viruses. Anti-viral agents are compounds which prevent infection of cells by viruses or replication of the virus within the cell. There are several stages within the process of viral infection which can be blocked or inhibited by anti-viral agents. These stages include, attachment of the virus to the host cell (immunoglobulin or binding peptides), uncoating of the virus (e.g. amantadine), synthesis or translation of viral in RNA (e.g. interferon), replication of viral RNA or DNA (e.g. nucleotide analogues), maturation of new virus proteins (e.g. protease inhibitors), and budding and release of the virus.

Anti-viral agents useful in the invention include but are not limited to: immunoglobulins, amantadine, interferons, nucleotide analogues, and protease inhibitors. Specific examples of anti-virals include but are not limited to Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscarnet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; and Zinviroxime.

Nucleotide analogues are synthetic compounds which are similar to nucleotides, but which have an incomplete or abnormal deoxyribose or ribose group. Once the nucleotide analogues are in the cell, they are phosphorylated, producing the triphosphate formed which competes with normal nucleotides for incorporation into the viral DNA or RNA. Once the triphosphate form of the nucleotide analogue is incorporated into the growing nucleic acid chain, it causes irreversible association with the viral polymerase and thus chain termination. Nucleotide analogues include, but are not limited to, acyclovir (used for the treatment of herpes simplex virus and varicella-zoster virus), gancyclovir (useful for the treatment of cytomegalovirus), idoxuridine, ribavirin (useful for the treatment of respiratory syncitial virus), dideoxyinosine, dideoxycytidine, zidovudine (azidothymidine), imiquimod, and resimiquimod.

The interferons are cytokines which are secreted by virus-infected cells as well as immune cells. The interferons function by binding to specific receptors on cells adjacent to the infected cells, causing the change in the cell which protects it from infection by the virus. α and β-interferon also induce the expression of Class I and Class II MHC molecules on the surface of infected cells, resulting in increased antigen presentation for host immune cell recognition. α and β-interferons are available as recombinant forms and have been used for the treatment of chronic hepatitis B and C infection. At the dosages which are effective for anti-viral therapy, interferons have severe side effects such as fever, malaise and weight loss.

Anti-fungal agents are used to treat superficial fungal infections as well as opportunistic and primary systemic fungal infections. Anti-fungal agents are useful for the treatment and prevention of infective fungi. Anti-fungal agents are sometimes classified by their mechanism of action. Some anti-fungal agents function, for example, as cell wall inhibitors by inhibiting glucose synthase. These include, but are not limited to, basiungin/ECB. Other anti-fungal agents function by destabilizing membrane integrity. These include, but are not limited to, immidazoles, such as clotrimazole, sertaconzole, fluconazole, itraconazole, ketoconazole, miconazole, and voriconacole, as well as FK 463, amphotericin B, BAY 38-9502, MK 991, pradimicin, UK 292, butenafine, and terbinafine. Other anti-fungal agents function by breaking down chitin (e.g. chitinase) or immunosuppression (501 cream).

Anti-parasitic agents kill or inhibit parasites. Examples of anti-parasitic agents, also referred to as parasiticides, useful for human administration include but are not limited to albendazole, amphotericin B, benznidazole, bithionol, chloroquine HCl, chloroquine phosphate, clindamycin, dehydroemetine, diethylcarbamazine, diloxanide furoate, eflornithine, furazolidaone, glucocorticoids, halofantrine, iodoquinol, ivermectin, mebendazole, mefloquine, meglumine antimoniate, melarsoprol, metrifonate, metronidazole, niclosamide, nifurtimox, oxamniquine, paromomycin, pentamidine isethionate, piperazine, praziquantel, primaquine phosphate, proguanil, pyrantel pamoate, pyrimethanmine-sulfonamides, pyrimethanmine-sulfadoxine, quinacrine HCl, quinine sulfate, quinidine gluconate, spiramycin, stibogluconate sodium (sodium antimony gluconate), suramin, tetracycline, doxycycline, thiabendazole, tinidazole, trimethroprim-sulfamethoxazole, and tryparsamide some of which are used alone or in combination with others.

Drug Formulations: Compositions useful in the practice of this invention can be formulated as pharmaceutical compositions together with pharmaceutically acceptable carriers for parenteral administration or enteral administration of for topical or local administration. For example, the compositions useful in the practice of the invention can be administered as oral formulations in solid or liquid form, or as intravenous, intramuscular, subcutaneous, transdermal, or topical formulation. Oral formulations are preferred.

The compositions are typically administered with pharmaceutically acceptable carriers. The term “pharmaceutically-acceptable carrier” as used herein means one or more compatible solid, or semi-solid or liquid fillers, diluants or encapsulating substances which are suitable for administration to a human or other mammal such as a dog, cat, horse, cow, sheep, or goat. The term “carrier” denotes an organic or inorganic ingredient, natural or synthetic, with which the active ingredient is combined to facilitate the application. The carriers are capable of being commingled with the preparations of the present invention, and with each other, in a manner such that there is no interaction which would substantially impair the desired pharmaceutical efficacy or stability. Carriers suitable for oral, subcutaneous, intravenous, intramuscular, etc. formulations can be found in Remington's Pharmaceutical Sciences, Mack Publishing Company, Easton, Pa.

Pharmaceutically acceptable carriers for oral administration include capsules, tablets, pills, powders, troches, and granules. In the case of solid dosage forms, the carrier can comprise at least one inert diluent such as sucrose, lactose or starch. Such carriers can also comprise, as is normal practice, additional substances other than diluents, e.g. lubricating agents such as magnesium stearate. In the case of capsules, tablets, troches and pills, the carrier can also comprise buffering agents. Carriers, such as tablets, pills and granules, can be prepared with coatings on the surfaces of the tablets, pills or granules which control the timing and/or the location of release of the pharmaceutical compositions in the gastrointestinal tract. In some embodiments, the carriers also target the active compositions to particular regions of the gastrointestinal tract and even hold the active ingredients at particular regions, such as is known in the art. Alternatively, the coated compounds can be pressed into tablets, pills, or granules. Pharmaceutically acceptable carriers include liquid dosage forms for oral administration, e.g. emulsions, solutions, suspensions, syrups and elixirs containing inert diluents commonly used in the art, such as water. Besides such inert diluents, compositions can also include adjuvants, such as wetting agents, emulsifying and suspending agents, and sweetening, flavoring agents.

The pharmaceutical preparations of the invention may be provided in particles. Particles as used herein means nano or microparticles (or in some instances larger) which can consist in whole or in part of the CO or CORM or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero order release, first order release, second order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the antagonist in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described by H. S. Sawhney, C. P. Pathak and J. A. Hubell in Macromolecules, (1993) 26:581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The invention provides methods for oral administration of a pharmaceutical composition of the invention. Oral solid dosage forms are described generally in Remington's Pharmaceutical Sciences, 18th Ed., 1990 (Mack Publishing Co. Easton Pa. 18042) at Chapter 89. Solid dosage forms for oral administration include capsules, tablets, pills, powders, troches or lozenges, cachets, pellets, and granules. Also, liposomal or proteinoid encapsulation can be used to formulate the present compositions (as, for example, proteinoid microspheres reported in U.S. Pat. No. 4,925,673). Liposomal encapsulation may include liposomes that are derivatized with various polymers (e.g., U.S. Pat. No. 5,013,556). In general, the formulation includes a compound of the invention and inert ingredients which protect against degradation in the stomach and which permit release of the biologically active material in the intestine.

In such solid dosage forms, the active compound is mixed with, or chemically modified to include, a least one inert, pharmaceutically acceptable excipient or carrier. The excipient or carrier preferably permits (a) inhibition of proteolysis, and (b) uptake into the blood stream from the stomach or intestine. In a most preferred embodiment, the excipient or carrier increases uptake of the compound, overall stability of the compound and/or circulation time of the compound in the body. Excipients and carriers include, for example, sodium citrate or dicalcium phosphate and/or (a) fillers or extenders such as starches, lactose, sucrose, glucose, cellulose, modified dextrans, mannitol, and silicic acid, as well as inorganic salts such as calcium triphosphate, magnesium carbonate and sodium chloride, and commercially available diluents such as FAST-FLO®, EMDEX®, STA-RX 1500®, EMCOMPRESS® and AVICEL®, (b) binders such as, for example, methylcellulose ethylcellulose, hydroxypropylmethyl cellulose, carboxymethylcellulose, gums (e.g., alginates, acacia), gelatin, polyvinylpyrrolidone, and sucrose, (c) humectants, such as glycerol, (d) disintegrating agents, such as agar-agar, calcium carbonate, potato or tapioca starch, alginic acid, certain silicates, sodium carbonate, starch including the commercial disintegrant based on starch, EXPLOTAB®, sodium starch glycolate, AMBERLITE®, sodium carboxymethylcellulose, ultramylopectin, gelatin, orange peel, carboxymethyl cellulose, natural sponge, bentonite, insoluble cationic exchange resins, and powdered gums such as agar, karaya or tragacanth; (e) solution retarding agents such a paraffin, (f) absorption accelerators, such as quaternary ammonium compounds and fatty acids including oleic acid, linoleic acid, and linolenic acid (g) wetting agents, such as, for example, cetyl alcohol and glycerol monosterate, anionic detergent surfactants including sodium lauryl sulfate, dioctyl sodium sulfosuccinate, and dioctyl sodium sulfonate, cationic detergents, such as benzalkonium chloride or benzethonium chloride, nonionic detergents including lauromacrogol 400, polyoxyl 40 stearate, polyoxyethylene hydrogenated castor oil 10, 50 and 60, glycerol monostearate, polysorbate 40, 60, 65, and 80, sucrose fatty acid ester, methyl cellulose and carboxymethyl cellulose; (h) absorbents, such as kaolin and bentonite clay, (i) lubricants, such as talc, calcium sterate, magnesium stearate, solid polyethylene glycols, sodium lauryl sulfate, polytetrafluoroethylene (PTFE), liquid paraffin, vegetable oils, waxes, CARBOWAX® 4000, CARBOWAX® 6000, magnesium lauryl sulfate, and mixtures thereof; (j) glidants that improve the flow properties of the drug during formulation and aid rearrangement during compression that include starch, talc, pyrogenic silica, and hydrated silicoaluminate. In the case of capsules, tablets, and pills, the dosage form also can comprise buffering agents.

Solid compositions of a similar type also can be employed as fillers in soft and hard-filled gelatin capsules, using such excipients as lactose or milk sugar, as well as high molecular weight polyethylene glycols and the like.

The solid dosage forms of tablets, dragees, capsules, pills, and granules can be prepared with coatings and shells, such as enteric coatings and other coatings well known in the pharmaceutical formulating art. They optionally can contain opacifying agents and also can be of a composition that they release the active ingredients(s) only, or preferentially, in a part of the intestinal tract, optionally, in a delayed manner. Exemplary materials include polymers having pH sensitive solubility, such as the materials available as EUDRAGIT® Examples of embedding compositions which can be used include polymeric substances and waxes.

Liquid dosage forms for oral administration include pharmaceutically acceptable emulsions, solutions, suspensions, syrups, and elixirs. In addition to the active compounds, the liquid dosage forms can contain inert diluents commonly used in the art, such as, for example, water or other solvents, solubilizing agents and emulsifiers, such as ethyl alcohol, isopropyl alcohol ethyl carbonate ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols, fatty acid esters of sorbitan, and mixtures thereof.

Besides inert diluents, the oral compositions also can include adjuvants, such as wetting agents, emulsifying and suspending agents, sweetening, coloring, flavoring, and perfuming agents. Oral compositions can be formulated and further contain an edible product, such as a beverage.

Suspensions, in addition to the active compounds, can contain suspending agents such as, for example ethoxylated isostearyl alcohols, polyoxyethylene sorbitol and sorbitan esters, microcrystalline cellulose, aluminum metahydroxide, bentonite, agar-agar, tragacanth, and mixtures thereof.

When used in its acid form, a compound of the present invention can be employed in the form of a pharmaceutically acceptable salt of the acid. Carriers such as solvents, water, buffers, alkanols, cyclodextrins and aralkanols can be used. Other auxiliary, non-toxic agents may be included, for example, polyethylene glycols or wetting agents.

The pharmaceutically acceptable carriers and compounds described in the present invention are formulated into unit dosage forms for administration to the patients. The dosage levels of active ingredients (i.e. compounds of the present invention) in the unit dosage may be varied so as to obtain an amount of active ingredient that is effective to achieve a therapeutic effect in accordance with the desired method of administration. The selected dosage level therefore mainly depends upon the nature of the active ingredient, the route of administration, and the desired duration of treatment. If desired, the unit dosage can be such that the daily requirement for an active compound is in one dose, or divided among multiple doses for administration, e.g. two to four times per day.

Compounds of the present invention also can be administered in the form of liposomes. As is known in the art, liposomes generally are derived from phospholipids or other lipid substances. Liposomes are formed by mono- or multi-lamellar hydrated liquid crystals that are dispersed in an aqueous medium. Any nontoxic, physiologically acceptable, and metabolizable lipid capable of forming liposomes can be used. The present compositions in liposome form can contain, in addition to a compound of the present invention, stabilizers, preservatives, excipients, and the like. The preferred lipids are the phospholipids and the phosphatidyl cholines (lecithins), both natural and synthetic. Methods to form liposomes are known in the art. See, for example, Prescott, Ed., Methods in Cell Biology, Volume XIV, Academic Press, New York, N.Y. (1976), p. 33, et seq.

Dosage forms for topical administration of a compound of this invention include powders, sprays, ointments, and inhalants as described herein. The active compound is mixed under sterile conditions with a pharmaceutically acceptable carrier and any needed preservatives, buffers, or propellants which may be required. Ophthalmic formulations, eye ointments, powders, and solutions also are contemplated as being within the scope of this invention.

Pharmaceutical compositions of the invention for parenteral injection comprise pharmaceutically acceptable sterile aqueous or nonaqueous solutions, dispersions, suspensions, or emulsions, as well as sterile powders for reconstitution into sterile injectable solutions or dispersions just prior to use. Examples of suitable aqueous and nonaqueous carriers, diluents, solvents, or vehicles include water ethanol, polyols (such as, glycerol, propylene glycol, polyethylene glycol, and the like), and suitable mixtures thereof; vegetable oils (such, as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions also can contain adjuvants such as preservatives, wetting agents, emulsifying agents, and dispersing agents. Prevention of the action of microorganisms can be ensured by the inclusion of various antibacterial and antifungal agents, for example, paraben, chlorobutanol, phenol sorbic acid, and the like. It also may be desirable to include isotonic agents such as sugars, sodium chloride, and the like. Prolonged absorption of the injectable pharmaceutical form can be brought about by the inclusion of agents which delay absorption, such as aluminum monostearate and gelatin.

Pharmaceutically acceptable carriers for intravenous administration include solutions containing pharmaceutically acceptable salts or sugars. Pharmaceutically acceptable carriers for intramuscular or subcutaneous injection include salts, oils, or sugars.

In some cases, in order to prolong the effect of the drug, it is desirable to slow the absorption of the drug from subcutaneous or intramuscular injection. This result can be accomplished by the use of a liquid suspension of crystalline or amorphous materials with poor water solubility. The rate of absorption of the drug then depends upon its rate of dissolution which, in turn, may depend upon crystal size and crystalline form. Alternatively, delayed absorption of a parenterally administered drug from is accomplished by dissolving or suspending the drug in an oil vehicle.

Injectable depot forms are made by forming microencapsule matrices of the drug in biodegradable polymers such a polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations also are prepared by entrapping the drug in liposomes or microemulsions which are compatible with body tissue.

The injectable formulations can be sterilized, for example, by filtration through a bacterial- or viral-retaining filter, or by incorporating sterilizing agents in the form of sterile solid compositions which can be dissolved or dispersed in sterile water or other sterile injectable medium just prior to use.

Also contemplated herein is pulmonary delivery of the compounds of the invention. The compound is delivered to the lungs of a mammal while inhaling, thereby promoting the traversal of the lung epithelial lining to the blood stream. See, Adjei et al., Pharmaceutical Research 7:565-569 (1990); Adjei et al., International Journal of Pharmaceutics 63:135-144 (1990) (leuprolide acetate); Braquet et al., Journal of Cardiovascular Pharmacology 13 (suppl.5): s.143-146 (1989)(endothelin-1); Hubbard et al., Annals of Internal Medicine 3:206-212 (1989)(α1-antitrypsin); Smith et al., J. Clin. Invest. 84:1145-1146 (1989) (α1-proteinase); Oswein et al., “Aerosolization of Proteins,” Proceedings of Symposium on Respiratory Drug Delivery II, Keystone, Colo., March, 1990 (recombinant human growth hormone); Debs et al., The Journal of Immunology 140:3482-3488 (1988) (interferon-γ and tumor necrosis factor α) and Platz et al., U.S. Pat. No. 5,284,656 (granulocyte colony stimulating factor).

Contemplated for use in the practice of this invention are a wide range of mechanical devices designed for pulmonary delivery of therapeutic products, including, but not limited to, nebulizers, metered dose inhalers, and powder inhalers, all of which are familiar to those skilled in the art.

Some specific examples of commercially available devices suitable for the practice of the invention are the ULTRAVENT® nebulizer, manufactured by Mallinckrodt, Inc., St. Louis, Mo.; the ACORN II® nebulizer, manufactured by Marquest Medical Products, Englewood, Colo.; the VENTOL® metered dose inhaler, manufactured by Glaxo Inc., Research Triangle Park, N.C.; and the SPINHALER® powder inhaler, manufactured by Fisons Corp., Bedford, Mass.

All such devices require the use of formulations suitable for the dispensing of a compound of the invention. Typically, each formulation is specific to the type of device employed and can involve the use of an appropriate propellant material, in addition to diluents, adjuvants, and/or carriers useful in therapy.

The composition is prepared in particulate form, preferably with an average particle size of less than 10 μm, and most preferably 0.5 to 5 μm, for most effective delivery to the distal lung.

Carriers include carbohydrates such as trehalose, mannitol, xylitol, sucrose, lactose, and sorbitol. Other ingredients for use in formulations may include lipids, such as DPPC, DOPE, DSPC and DOPC, natural or synthetic surfactants, polyethylene glycol (even apart from its use in derivatizing the inhibitor itself), dextrans, such as cyclodextran, bile salts, and other related enhancers, cellulose and cellulose derivatives, and amino acids.

The use of liposomes, microcapsules or microspheres, inclusion complexes, or other types of carriers is also contemplated.

Formulations suitable for use with a nebulizer, either jet or ultrasonic, typically comprise a compound of the invention dissolved in water at a concentration of about 0.1 to 25 mg of biologically active protein per mL of solution. The formulation also can include a buffer and a simple sugar (e.g., for protein stabilization and regulation of osmotic pressure). The nebulizer formulation also can contain a surfactant to reduce or prevent surface-induced aggregation of the inhibitor composition caused by atomization of the solution in forming the aerosol.

Formulations for use with a metered-dose inhaler device generally comprise a finely divided powder containing the inhibitor compound suspended in a propellant with the aid of a surfactant. The propellant can be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid also can be useful as a surfactant.

Formulations for dispensing from a powder inhaler device comprise a finely divided dry powder containing the inhibitor and also can include a bulking agent, such as lactose, sorbitol, sucrose, mannitol, trehalose, or xylitol, in amounts which facilitate dispersal of the powder from the device, e.g., 50 to 90% by weight of the formulation.

Nasal delivery of the compounds and composition of the invention also is contemplated. Nasal delivery allows the passage of the compound or composition to the blood stream directly after administering the therapeutic product to the nose, without the necessity for deposition of the product in the lung. Formulations for nasal delivery include those with dextran or cyclodextran. Delivery via transport across other mucous membranes also is contemplated.

Compositions for rectal or vaginal administration are preferably suppositories which can be prepared by mixing the compounds of the invention with suitable nonirritating excipients or carriers, such as cocoa butter, polyethylene glycol, or suppository wax, which are solid at room temperature, but liquid at body temperature, and therefore melt in the rectum or vaginal cavity and release the active compound.

In order to facilitate delivery of compounds across cell and/or nuclear membranes, compositions of relatively high hybrophobicity are preferred. Compounds can be modified in a manner which increases hydrophobicity, or the compounds can be encapsulated in hydrophobic carriers or solutions which result in increased hydrophobicity.

The compounds and pharmaceutical compositions of the invention can be administered to a subject by any suitable route. For example, the compositions can be administered orally, including sublingually, rectally, parenterally, intracisternally, intravaginally, intraperitoneally, topically and transdermally (as by powders, ointments, or drops), bucally, or nasally. The term “parenteral” administration as used herein refers to modes of administration other than through the gastrointestinal tract, which include intravenous, intramuscular, intraperitoneal, intrasternal, intramammary, intraocular, retrobulbar, intrapulmonary, intrathecal, subcutaneous and intraarticular injection and infusion. Surgical implantation also is contemplated, including, for example, embedding a composition of the invention in the body such as, for example, in the brain, in the abdominal cavity, under the splenic capsule, brain, or in the cornea. In some preferred embodiments, the compounds are administered orally, topically, intravenously or intramuscularly.

Preferably, the compounds are administered orally. The preferred dose levels will be determined in animals for representative compounds. All CORM compounds described in the present invention generate CO after administration to the body. The CO generated will bind to hemoglobin in red blood cells. Thus, dose finding studies will initially be guided by measurement of carboxyhemoglobin (COHb) levels in the blood. Methods for the measurement of COHb levels in the blood are well known and are being used on a regular basis in diagnostic laboratories. In normal healthy humans COHb levels are about 0.5% in healthy nonsmokers and up to 9% in smokers. Preferred dose levels of the compounds described in the present invention are such that no significant rise in COHb levels is observed. However, in some applications a transient rise in COHb levels up to 10% may be tolerated. This level of COHb is not associated with any symptoms.

As representative examples, compounds in Classes 1 and 4 are administered in a dosage ranging between 5 and 25 mmol/day depending on the nature of the CO containing compound and its molar CO content. The same range of dosage of the CO containing molecule is applied for Class 3 compounds. For conjugates in classes 2 and 5, the dose can vary from a lower 5 mg/day up to 10 g day with preferred values in the range of 1 g/day for adults. These are indicative values dependent on the nature of the CO carrier molecular fragment and comply with the usual ranges for drug or agent dosage. For the polyhalomethane and similar compounds in Class 6, e.g., dichloromethane, the dose range varies between 0.01 to 10 mmol/kg per os, with a preferred dose level of 0.1 mmol/kg. The same range of dosage of active principle is applied in the Class 7 compounds.

In general, dosage is adjusted appropriately to achieve desired drug levels, locally or systemically. In the event that the response in a subject is insufficient at such doses, even higher doses (or effective higher doses by a different, more localized delivery route) may be employed to the extent that patient tolerance permits.

The pharmaceutical preparations of the invention, when used in alone or together with other agents are administered in therapeutically effective amounts. A therapeutically effective amount will be that amount which establishes a level of the drug(s) effective for treating a subject, such as a human subject. An effective amount means that amount alone or with multiple doses, necessary to delay the onset of, inhibit completely or lessen the progression of or halt altogether the onset or progression of an infection. This can be monitored by routine diagnostic methods known to those of ordinary skill in the art. When administered to a subject, effective amounts will depend, of course, on the particular side effect chosen as the end-point; the severity of the condition; individual patient parameters including age, physical condition, size and weight; concurrent treatment; frequency of treatment; and the mode of administration. These factors are well known to those of ordinary skill in the art and can be addressed with no more than routine experimentation.

The factors involved in determining an effective amount are well known to those of ordinary skill in the art and can be addressed with no more than routine experimentation. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

According to another aspect of the invention, medical products are provided. The medical product includes a CORM containing vial and, optionally, a vial containing another agent (e.g., an anti-infective agent). The medical product also includes indicia indicating that the CORM is for inhibiting an infection. The indicia can be on a label attached to the CORM containing vial or can be in a package contain the CORM containing vial.

The methods of the invention have important implications for patient treatment and also for the clinical development of new therapies. It is also expected that clinical investigators now will use the present methods for determining entry criteria for human subjects in clinical trials. Health care practitioners select therapeutic regimens for treatment based upon the expected net benefit to the subject. The net benefit is derived from the risk to benefit ratio.

The entire disclosure of any patent or published application referred to herein is incorporated herein by reference in its entirety.

The invention is exemplified by the following Examples and is illustrated herein in reference to treatment of certain types of infections. In these illustrative treatments, standard state-of-the-art models have been used.

### EXAMPLES

### Example 1

### Introduction

Carbon monoxide (CO) is a colorless and odorless diatomic gas, chemically inert, that occurs in nature as a product of oxidation or combustion of organic matter. Owing to its lethal effect when present in high concentrations, CO was considered for many years to be only an environmental toxicant that results from air pollution by automobile exhaust. The knowledge that the human body is able to produce small quantities of CO and the evidence that CO derived from heme oxygenase activity contributes to important intracellular functions have modified our perception of CO as a pernicious toxin to include its beneficial effects (15, 16, 22). In consequence, the application of CO gas or CO releasing molecules (CORMs) has emerged as a new therapeutic strategy in medicine (10, 13, 18). The evolution of CO from a toxicant to a molecule of mounting importance in mammals finds a parallel in another diatomic molecule, nitric oxide (NO) (17). NO is produced in the body by the nitric oxide synthase and shares with CO many downstream signaling pathways and regulatory functions, in particular, those associated with the activation of soluble guanylyl cyclase (7, 8, 12). In addition, there is an interplay between the two molecules, since it is proposed that CO is a modulator of nitric oxide synthase (10, 22) and NO up-regulates heme oxygenase (19, 20), which in turn catalyzes the oxidative degradation of free heme into biliverdin, with the concomitant release of iron and CO. NO also constitutes one of the weapons that the mammalian immune system uses to fight pathogens (3, 4). The bactericidal function of NO relies on the deleterious effects caused in the pathogen, e.g., the nitrosylation of iron centers. Although CO is a stable neutral molecule with a long half-life, it shares with NO the high affinity for iron of heme proteins, which is the basis of its toxicity. We therefore set out to explore the possible action of CO on bacterial growth rates. For this purpose, we tested the bioactivity of CO, applied either in the gaseous form or via treatment with CORMs, on Escherichia coli and Staphylococcus aureus. These bacteria are major human pathogens that are widespread in the community and are responsible for hospital-acquired infections, exhibiting a concerning degree of antibiotic resistance.

### Materials and Methods:

Reagents: The different sources or references for CO were as follows: tricarbonyldichlororuthenium(II) dimer (CORM-2), Sigma; tricarbonylchloro(glycinato)ruthenium(II) (CORM-3), reference 6; bromo(pentacarbonyl)manganese (compound of Formula IV), reference 5; and tetraethylammonium molybdenum pentacarbonyl bromide (compound of Formula V), reference 2. All compounds were freshly prepared as 10 mM stock solutions by dissolution in dimethyl sulfoxide, pure distilled water, or methanol.

Bacterial strains and growth conditions: E. coli K-12 ATCC 23716 and S. aureus NCTC8325 were grown in minimal salts (MS) medium (1.3% [wt/vol] Na₂HPO₄, 0.3% [wt/vol] KH₂PO₄, 0.05% [wt/vol] NaCl, and 0.1% [wt/vol] NH₄Cl supplemented with 20 mM glucose, 2 mM MgSO₄, 100 μM CaCl₂, and 0.25% [wt/vol] Casamino Acids) and in Luria-Bertani (LB) medium (1% [wt/vol] tryptone, 0.5% [wt/vol] yeast extract, and 1% [wt/vol] NaCl), respectively, under different oxygen supply conditions. Aerobic experiments were undertaken with flasks filled to one-fifth of their volume, microaerobic tests were conducted with closed flasks filled to one-half of their volume, and anaerobic conditions were produced in rubber-sealed flasks that, once filled with medium and closed, were extensively fluxed with nitrogen gas.

CO gas and CORM treatment: Overnight cultures of E. coli or S. aureus grown in LB or tryptic soy broth, respectively, were used to inoculate fresh MS medium (E. coli) or LB medium (S. aureus), and the cultures on fresh medium were incubated at 37° C. under the required aeration conditions to an optical density at 600 um of 0.3. At this point, cells were exposed to a flux of CO gas for 15 min or to CORMs. Untreated cells were bubbled with nitrogen gas or treated with dimethyl sulfoxide, water, or methanol, depending on the solvent used to dissolve the CORM. The inactive form of compound of Formula V was prepared by mixing vigorously with 20% methanol in a closed flask over 2 to 3 h. The counterion of compound of Formula V, tetraethyl ammonium bromide, and one of the products of compound of Formula V decomposition, sodium molybdate, were used at the same concentration as compound of Formula V (50 μM).

Viability assays: The number of viable cells was evaluated by measuring the CFU per milliliter upon plating serial dilutions of the various cultures onto agar plates. The percent survival was calculated as the number of colonies originated by treated cultures divided by the number of colonies formed upon the plating of untreated cultures. Sensitivity tests were conducted by plating 5 μl serial dilutions of cultures grown for 4 h and treated with CORMs, with or without the CO scavenger hemoglobin (Hb [bovine form used at 20 μM; Sigma]), onto agar. The experiments were performed with a minimum of three independent cultures, and the results are presented in the figures as averaged values with error bars representing one standard deviation.

The investigation of MICs and minimal bactericidal concentrations (MBCs) was carried out by the tube dilution test. Briefly, 2.5 ml of minimal medium was inoculated with an overnight culture of E. coli or S. aureus to give an optical density at 600 nm of 0.005 to 0.01. Different concentrations of CORM-2, between 150 μM and 2 mM, were added to the diluted suspensions in the wells of 24-well plates, and the plates were incubated for at least 18 h at 37° C. and 90 rpm. The concentration of CORM-2 in the first well in the series with no sign of visible growth was reported as the MIC. All the cultures that exhibited a lack of cell growth were then subsequently plated onto agar devoid of any drug. After incubation at 37° C. for 24 h, the lowest concentration of CORM-2 in a culture with no growth was assumed to be the MBC.

CO release kinetics: CORMs were mixed with MS or LB medium in sealed vessels, and the vessels were incubated at room temperature under constant stirring and protected from light. Gas samples were collected after 30 min and 4 h and analyzed using a gas chromatograph (Thermo Finnigan Trace) equipped with a CTRI column (Alltech) and a thermal conductivity detector. The CO released was quantified using a calibration curve recorded prior to the reaction course.

Inductively coupled plasma mass spectrometry analysis: E. coli cells cultured in MS medium with or without 50 μM of compound of Formula V were collected after 1 h of growth, and the cellular metal content was analyzed at Instituto de Investigacao das Pescas e do Mar, Lisbon, Portugal. The intracellular concentration of Mo in E. coli cultures was assayed on a quadropole inductively coupled plasma mass spectrometer (X series; Thermo Elemental) equipped with a Peltier impact bead spray chamber and a concentric Meinhard nebulizer. The experimental parameters were as follows: 790 W of forward power, peak jumping mode, and 150 sweeps per replicate (dwell time, 10 ms; dead time, 30 ns). A seven-point calibration within a range of 1 to 100 μg liter⁻¹ was used to quantify metal concentrations. Coefficients of variation for determinations of metal content (n=5) ranged between 0.5 and 2%. The precision and accuracy of metal concentration measurements, as determined through the repeated analysis of reference materials (TORT-1, TORT-2, DORM-2, and DORM-3 from the National Research Council of Canada) by using indium as an internal standard, were within 1 to 2%. Procedural blanks always accounted for less than 1% of the total molybdenum concentrations in the samples.

### Results and Discussion:

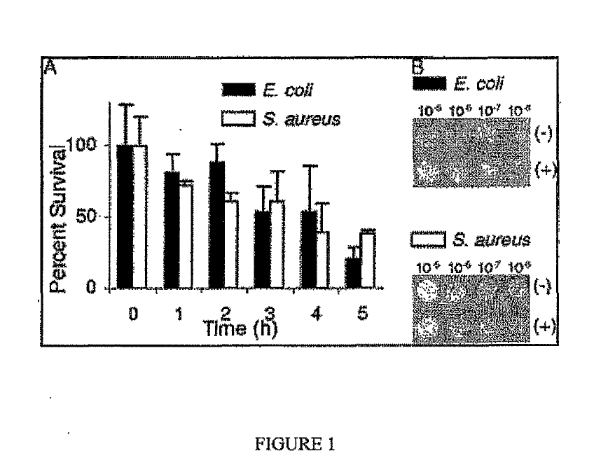

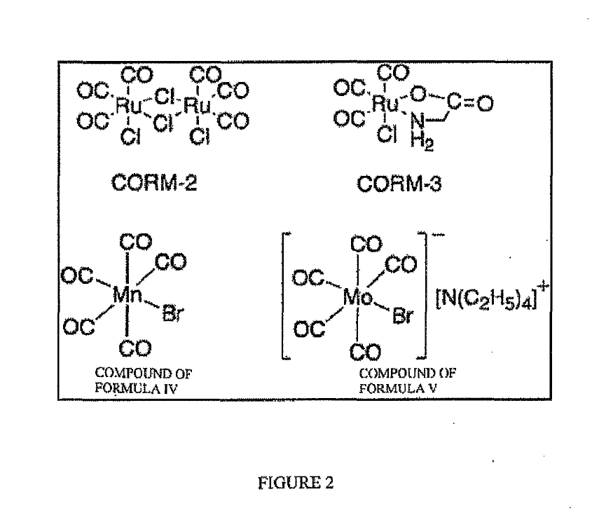

The effect of CO on the viability of bacteria was investigated first by the direct delivery of CO gas. The administration of CO gas, fluxed into the growing cultures, led to a significant growth impairment of E. coli and S. aureus (FIG. 1). To evaluate the potential of CORMs, the compounds indicated in FIG. 2 were selected. CORM-2 and CORM-3 are active in a variety of CO-mediated biological processes, both in vitro and in vivo (9).

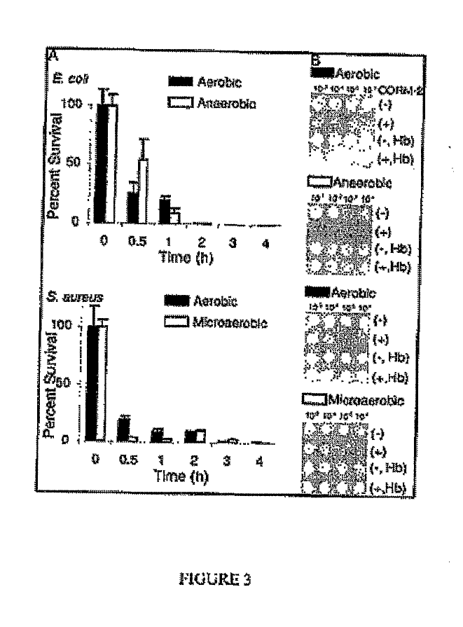

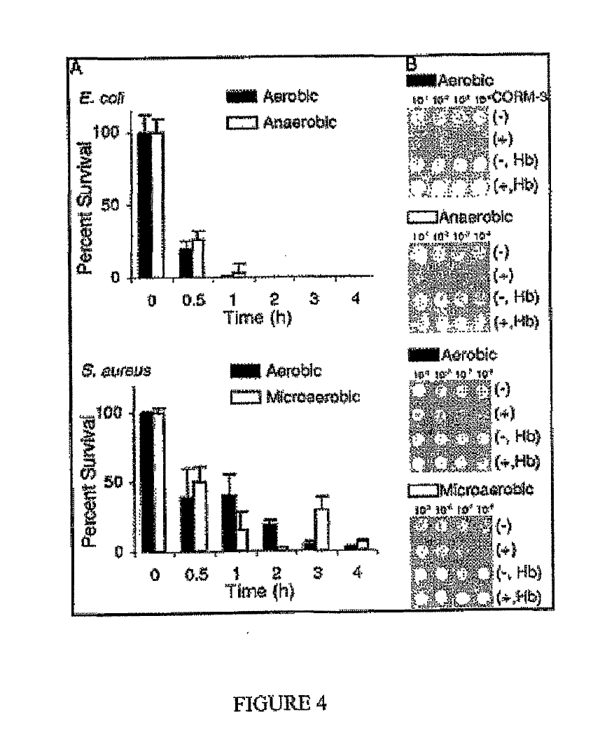

In the first series of experiments, the effect of CO released from CORM-2 on the growth of E. coli and S. aureus was studied with bacteria cultured under different levels of oxygen supply. Shortly after the exposure to CORM-2, the percentage of surviving cells significantly diminished (FIG. 3). Experiments using water-soluble CORM-3 revealed that, albeit requiring higher concentrations than CORM-2 due to its chemical composition, the compound also strongly decreased the viability of E. coli and S. aureus cells (FIG. 4). However, while the addition of CORM-3 resulted in a strong inhibition of E. coli cell growth, S. aureus was more resistant to CORM-3 (FIG. 4A), particularly under aerobic conditions. In general, the action of the two compounds was rapid and extended over time, as cells did not resume growth over the subsequent 4 h (FIGS. 3 and 4) or after 8 h (data not shown).

In order to examine whether the bactericidal effect of CORMs was due to CO, cell growth experiments with CORMs were also performed in the presence of Hb, a high-affinity CO scavenger. In all cases, the bactericidal effect on E. coli and S. aureus was completely lost (FIGS. 3B and 4B), thus demonstrating that the antimicrobial action of CORMs is dependent on their release of CO.

Bactericidal activity has been defined as a ratio of the MBC to the MIC of <4 (14). The determination of the CORM-2 MBC/MIC ratios for E. coli and S. aureus to be 1.5 and 1.0, respectively, revealed the bactericidal character of CORM-2.

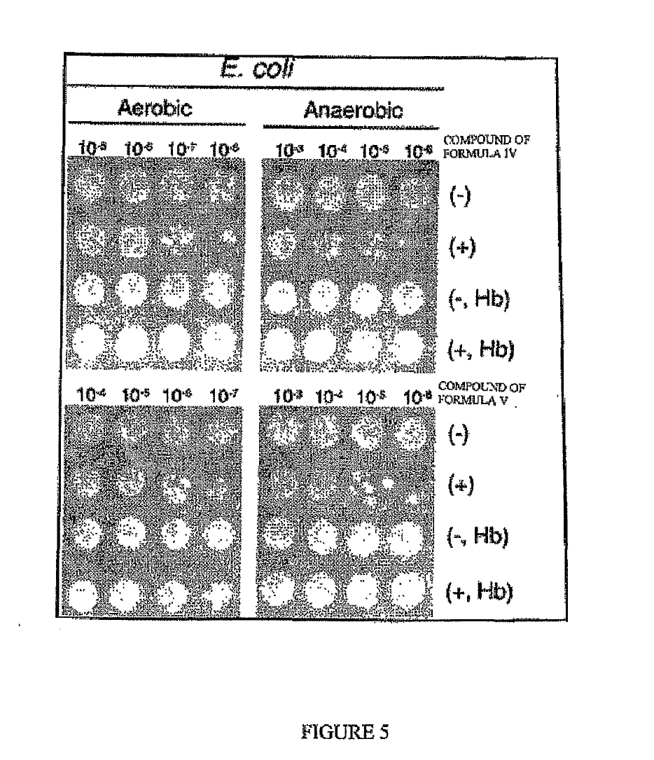

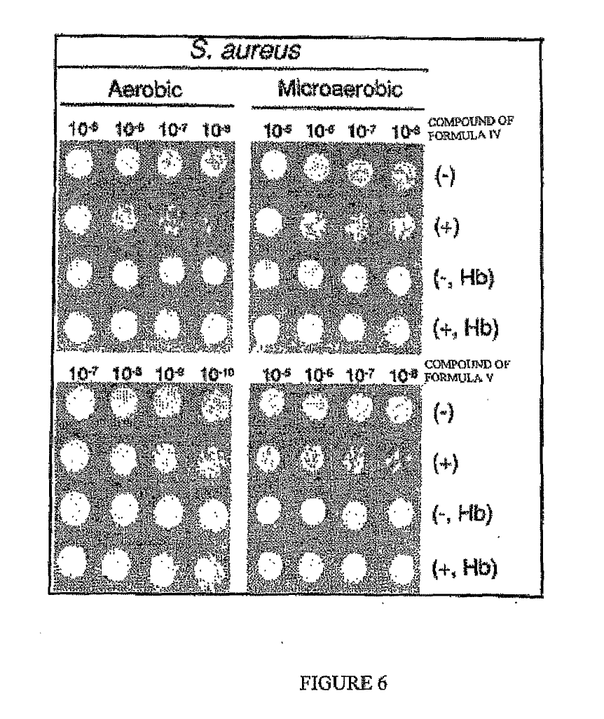

The two other CORMs used to investigate the bactericidal effect of CO, namely, manganese carbonyl compound of Formula IV and molybdenum carbonyl compound of Formula V, were also seen to be capable of strongly reducing the viability of E. coli and S. aureus (FIGS. 5 and 6). Again, the addition of Hb completely eliminated the harmful action of compound of Formula IV and compound of Formula V on the two bacteria (FIGS. 5 and 6). Furthermore, to ensure that the activity of compound of Formula V was not related to its decomposition products, we tested the effects of tetraethyl ammonium bromide, sodium molybdate, and a solution of inactivated compound of Formula V, obtained after the cessation of CO release on bacterial growth (see Materials and Methods). None of these compounds had bactericidal properties or altered growth kinetics (data not shown). Therefore, the bactericidal effects of compound of Formula V are due to its capacity to release CO.

It should be mentioned that neither CORM-2 nor CORM-3 releases CO gas when dissolved in the media utilized, even at concentrations higher than those used in our experiments (Table 1). Furthermore, although compound of Formula IV and compound of Formula V release CO gas upon dissolution in the medium, they do so in rather small amounts within the time scale of the experiment (Table 1). However, inductively coupled plasma mass spectrometry analysis of E. coli cells incubated with compound of Formula V revealed a very large increase in the content of Mo (155 μg g⁻¹) compared to that in control cells (2.5 μg g⁻¹), confirming that the Mo from compound of Formula V accumulates inside the E. coli cells, where it releases CO to the cellular targets.

TABLE 1
CO released into medium by CORMs^{α}

| | CO equivalent in MS | | CO equivalent in LB | |
|---|---|---|---|---|
| | medium at: | | medium at: | |
| CORM (concn, mM) | 30 min | 240 min | 30 min | 240 min |
| CORM-2 (5) | 0 | 0 | 0 | 0.1 |
| CORM-3 (12) | 0 | 0 | 0 | 0 |
| COMPOUND OF | 0 | 0.5 | 0 | 0.5 |
| FORMULA IV (6) | | | | |
| COMPOUND OF | 1.4 | 3.8 | 0.2 | 1.6 |
| FORMULA V (6) | | | | |

^{α}Amounts of CO are expressed as CO equivalents (number of CO groups released per CORM molecule).

Since the bactericidal effect of the CORMs does not require the release of CO gas to the extracellular medium (Table 1), we must conclude that CO has to be delivered to the cellular targets directly from the CO-RMs. Because Mo from bactericidally active (CO-loaded) Compound of Formula V is found to accumulate rapidly within cells, we infer that it transports CO and delivers it into the intracellular space, where it reaches the cellular targets and causes the decrease of bacterial cell viability. If Hb is present in the medium, the high affinity of Hb for CO results in a fast transfer (or abstraction) of the active CO from the CORMs (or from gas) to the protein hemes and the effective scavenging of CO as COHb (see below). Under these conditions, no CO will be available for intracellular delivery and the cells remain alive.

Albeit with some minor deviations, the general pattern of our results shows that CORM toxicity is enhanced when growth occurs under lower oxygen concentrations. For example, compound of Formula IV was more effective in reducing the viability of E. coli cells grown anaerobically (200 μM compound of Formula IV) than that of cells grown aerobically (500 μM compound of Formula IV). The augmentation of the effect of CO at low oxygen concentrations may be explained by the preferential binding of CO to the ferrous form of heme proteins, which are predominant under reducing environments. More importantly, the bactericidal effect of CORMs under anaerobic conditions indicates that growth inhibition is not restricted to the impairment of the respiratory chain by the binding of CO to cytochrome oxidase, which is likely to contribute to the bactericidal activity of these compounds under aerobic conditions. This fact is quite important since pathogen colonization occurs in near-anaerobic environments and since many pathogens are anaerobic organisms. On the other hand, the type of bacterial cell wall also seems not to interfere with the action of CORMs, as judged by the similar decreases in cell viability observed for the gram-positive (S. aureus) and gram-negative (E. coli) species upon treatment with the same CORM. Hence, CORMs have the potential, for use as bactericides and anti-infective against a wide range of microorganisms independently of the type of cell wall and oxygen growth requirements.

The difference between the degrees of action of dissolved molecular CO gas and CORMs is striking. When administered as gas, CO had to be present in rather high concentrations (ca. 1 mM) to become effective as a bactericide. The ability of CORMs to accumulate inside bacterial cells before they release CO makes these compounds highly effective CO donors to bacterial targets, thereby strongly enhancing the bactericidal efficacy of CO. In fact, the CORMs used in this study were able to transfer CO to Hb to form COHb, as judged by the shift of the Hb Soret band from 413 to 418 nm (data not shown) and by the results depicted in FIGS. 3B, 4B, 5, and 6. Hence, CORMs are capable of delivering CO to heme-containing molecules, as had been shown before for the rapid carbonylation of myoglobin by CORM-3 (11). Likewise, the carbonylation of Hb by CORM-2 and CORM-3 occurs within the mixing time, while that by compound of Formula IV and compound of Formula V takes place in less than 15 min.

It is well known that the biological effect of CO on mammalian cells is due mainly to its interaction with iron-containing proteins, such as the above-mentioned cytochrome oxidase. In addition to heme proteins and sensors, CO may bind to almost all transition metal-containing proteins. Without intending to be bound by any particular mechanism or theory, it is believed that CO may bind to transition metal-containing proteins in microorganisms (such as bacteria), giving rise to structural modifications and alterations of their biological functions and possibly accounting for the toxic effect of CO on the microorganisms revealed in this study.

In spite of the increasing expectations for the use of CO in medicine (10, 13, 18), until now, the role of CO as a bactericidal compound had remained unexplored. Nevertheless, in the early 1970s it was reported that the addition of CO to an aerobic culture of E. coli caused a decrease in DNA replication (21). However, as the authors of the study did not observe any effect of CO on cells growing anaerobically on glucose, they concluded that the inhibition of DNA synthesis in cells grown under aerobic conditions was not due to a direct effect on the replication apparatus but resulted from indirect effects, such as ATP or deoxynucleoside triphosphate depletion (21). In more recent years, in spite of several public concerns, CO has been used by the food industry to generate the bright red color of the dark muscle tissue of meat and fish, which results from the great affinity of CO for the Fe (II) binding site of myoglobin. Interestingly, a very recent study of the influence of different packing systems on meat preservation indicated that packages to which CO gas had been added exhibited less bacterial growth than other packages. These results suggest that CO may be one of the packaging gases responsible for the inhibition of the growth of microorganisms (1). We now show that CO and, in particular, CORMs have the ability to kill bacteria under aerobic and anaerobic conditions. We submit that CORMs constitute a novel class of anti-infective (e.g., antibacterial) molecules that may be used to deliver CO to targets of infection (e.g., bacterial infection), and avoid the in vivo scavenging of CO by the red blood cells (10). In particular, nonsystemic anti-infectives (e.g., antibacterial agents) may be a relatively easy application for CORMs. Anti-infective agents (e.g., antibacterial agents) based upon completely new concepts are urgently required, as the emergence and spread of drug-resistant bacterial pathogens reveal a concerning decrease in the effectiveness of currently available antibiotics.

### REFERENCES

1. Brooks, J. C., M. M. Brashears, M. F. Miller, A. R. Hoyle, J. D. Kellermeier, and J. M. Mehaffey. 2006. The spoilage characteristics of ground beef packaged in high-oxygen and low-oxygen modified atmosphere packages, p. 61-65. In Proceedings of the Reciprocal Meat Conference. University of Illinois at Urbana-Champaign, Urbana-Champaign.
2. Burgmayer, S. J. N., and J. L. Templeton. 1985. Synthesis and structure of a 7-coordinate molybdenum carbonyl fluoride derivative, Et₄N Mo(CO)₂ (S₂CNEt₂)₂F. Inorg. Chem. 24:2224-2230.
3. Chakravortty, D., and M. Hensel. 2003. Inducible nitric oxide synthase and control of intracellular bacterial pathogens. Microbes Infect. 5:621-627.
4. Fang, F. C. 2004. Antimicrobial reactive oxygen and nitrogen species: concepts and controversies. Nat. Rev. Microbiol. 2:820-832.
5. Herrmann, W. A., and G. Brauer (ed.). 1997. Synthetic methods of organometallic and inorganic chemistry, vol. 7. Georg Thieme Verlag, Stuttgart, Germany.
6. Johnson, T. R., B. E. Mann, I. P. Teasdale, H. Adams, R. Foresti, C. J. Green, and R. Motterlini. 2007. Metal carbonyls as pharmaceuticals? [Ru(CO)₃Cl(glycinate)], a CO-releasing molecule with an extensive aqueous solution chemistry. Dalton Trans. 15:1500-1508.
7. Kharitonov, V. G., V. S. Sharma, R. B. Pilz, D. Magde, and D. Koesling. 1995. Basis of guanylate cyclase activation by carbon monoxide. Proc. Natl. Acad. Sci. USA 92:2568-2571.
8. Moncada, S., and E. A. Higgs. 2006. The discovery of nitric oxide and its role in vascular biology. Br. J. Pharmacol. 147(Suppl. 1):S193-S201.
9. Motterlini, R., J. E. Clark, R. Foresti, P. Sarathchandra, B. E. Mann, and C. J. Green. 2002. Carbon monoxide-releasing molecules: characterization of biochemical and vascular activities. Circ. Res. 90:E17-E24.
10. Motterlini, R., B. E. Mann, and R. Foresti. 2005. Therapeutic applications of carbon monoxide-releasing molecules. Expert Opin. Investig. Drugs 14:1305-1318.
11. Motterlini, R., P. Sawle, J. Hammad, S. Bains, R. Alberto, R. Foresti, and C. J. Green. 2005. CORM-A1: a new pharmacologically active carbon monoxide-releasing molecule. FASEB J. 19:284-286.
12. Mungrue, I. N., D. S. Bredt, D. J. Stewart, and M. Husain. 2003. From molecules to mammals: what's NOS got to do with it? Acta Physiol. Scand. 179:123-135.
13. Nakao, A., A. M. Choi, and N. Murase. 2006. Protective effect of carbon monoxide in transplantation. J. Cell. Mol. Med. 10:650-671.
14. Pankey, G. A., and L. D. Sabath. 2004. Clinical relevance of bacteriostatic versus bactericidal mechanisms of action in the treatment of Gram-positive bacterial infections. Clin. Infect. Dis. 38:864-870.
15. Piantadosi, C. A. 2002. Biological chemistry of carbon monoxide. Antioxid. Redox Signal. 4:259-270.
16. Ryter, S. W., J. Alam, and A. M. Choi. 2006. Heine oxygenase-1/carbon monoxide: from basic science to therapeutic applications. Physiol. Rev. 86:583-650.
17. Ryter, S. W., D. Morse, and A. M. Choi. 2004. Carbon monoxide: to boldly go where NO has gone before. Sci. STKE 2004:RE6.
18. Ryter, S. W., and L. E. Otterbein. 2004. Carbon monoxide in biology and medicine. Bioessays 26:270-280.
19. Ryter, S. W., L. E. Otterbein, D. Morse, and A. M. Choi. 2002. Heme oxygenase/carbon monoxide signaling pathways: regulation and functional significance. Mol. Cell. Biochem. 234-235:249-263.
20. Srisook, K., C. Kim, and Y. N. Cha 2005. Role of NO in enhancing the expression of HO-1 in LPS-stimulated macrophages. Methods Enzymol. 396:368-377.
21. Weigel, P. H., and P. T. Englund. 1975. Inhibition of DNA replication in Escherichia coli by cyanide and carbon monoxide. J. Biol. Chem. 250:8536-8542.
22. Wu, L., and R. Wang. 2005. Carbon monoxide: endogenous production, physiological functions, and pharmacological applications. Pharmacol. Rev. 57:585-630.

### Example 2

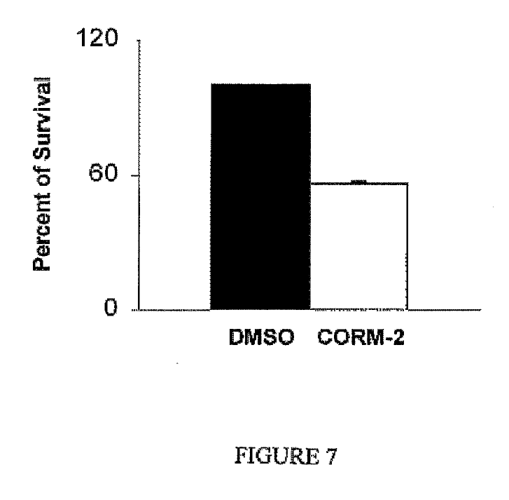

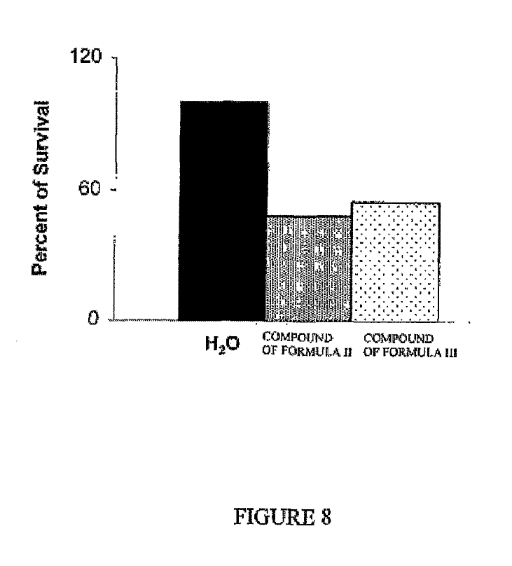

The bactericidal effect of CORMs on Helicobacter pylori (H. pylori) was evaluated by using diffusion disks and measuring the inhibition halos. Briefly, H. pylori 26695 was grown on blood agar plates for 24 hours at 37° C. in a microaerobic atmosphere (Genbox microaer, BioMerieux). The bacteria were removed from plates, resuspended in 3 ml of Brucella Broth (BB) and 200 μl of this suspension was inoculated in blood agar plates. Then, the paper disks were placed in the center of the inoculated plates and 15 μl of each CORM was added. The CORMs used in this assays were CORM-2 and 2 others water soluble CORMs: compound of Formula II and the compound of Formula III. The plates were incubated in the same conditions described during 24-36 hours. Afterwards, the inhibition halos were measured and the assays were repeated at least 2 times and average values were reported. FIGS. 7 and 8 show that CORM-2, compound of Formula II, and compound of Formula III have a bactericidal effect on H. pylori.

1. A method for treating a subject having or at risk of having an infection comprising:
administering to a subject in need of such a treatment an effective amount of carbon monoxide (CO) to treat the infection.

2. The method of claim 1, wherein the CO is administered as a CORM.

3. (canceled)

4. The method of claim 1, wherein the subject is otherwise free of indications calling for treatment with the CO.

5. The method of claim 2 wherein the CORM is an organometallic compound or an organic compound.

6.-9. (canceled)

10. A method of treatment of an infection comprising:
instructing a subject having or at risk of having an infection to take an effective amount of CO for the purpose of treatment of the infection.

11. The method of claim 10, where the subject is instructed to take the effective amount of CO in the form of a CORM.

12. The method of claim 11, wherein the subject is instructed to take the CORM orally.

13.-15. (canceled)

16. A medical treatment product comprising a package containing a CORM and containing indicia indicating that the CORM is for treating an infection.

17.-20. (canceled)

21. A compound having a structure: 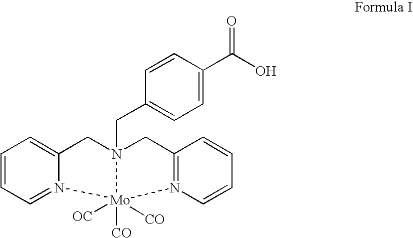
or a salt thereof.

22. A compound having a structure: 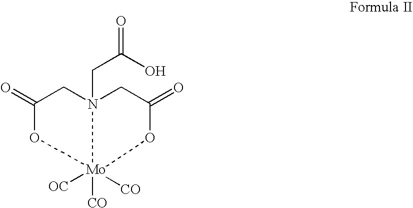
or a salt thereof.

23. A compound having a structure: 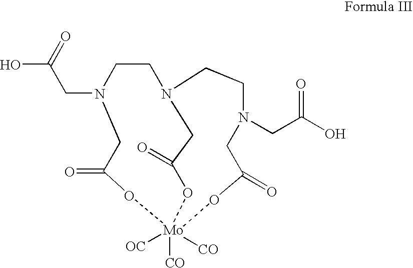
or a salt thereof.

24. A pharmaceutical composition comprising a compound of claim 21 and a pharmaceutically acceptable carrier.

25. (canceled)

26. A method for treating a subject having Helicobacter pylori infection comprising:
administering to a subject in need of such a treatment a composition of claim 24 in an effective amount to treat the Helicobacter pylori infection.

27. (canceled)

28. The method of claim 26, wherein the composition is administered orally.

29. A pharmaceutical composition comprising a compound of claim 22 and a pharmaceutically acceptable carrier.

30. A pharmaceutical composition comprising a compound of claim 23 and a pharmaceutically acceptable carrier.

31. A method for treating a subject having Helicobacter pylori infection comprising:
administering to a subject in need of such a treatment a composition of claim 29 in an effective amount to treat the Helicobacter pylori infection.

32. A method for treating a subject having Helicobacter pylori infection comprising:
administering to a subject in need of such a treatment a composition of claim 32 in an effective amount to treat the Helicobacter pylori infection.

33. The method of claim 31, wherein the composition is administered orally.

34. The method of claim 32, wherein the composition is administered orally.
